(19) United States
(12) Patent Application Publication
(10) Pub. No.: US 2026/0007864 A1
(43) Pub. Date: Jan. 8, 2026

(54) INFLATABLE INTRODUCER SHEATH

(71) Applicant: EDWARDS LIFESCIENCES CORPORATION, Irvine, CA (US)

(72) Inventor: Sagi Libovitch, Sdot Yam (IL)

(73) Assignee: EDWARDS LIFESCIENCES CORPORATION, Irvine, CA (US)

(21) Appl. No.: 19/325,875

(22) Filed: Sep. 11, 2025

Related U.S. Application Data
(63) Continuation of application No. PCT/US2024/016761, filed on Feb. 21, 2024.
(60) Provisional application No. 63/454,415, filed on Mar. 24, 2023.

(51) Int. Cl. A61M 25/06; A61M 25/00

(52) U.S. Cl. CPC A61M 25/0662; A61M 25/0045; A61M 2025/0004; A61M 2025/0063; A61M 2205/0216; A61M 2205/3379

(57) ABSTRACT

Various implementations include an inflatable sheath for delivering a medical device, the sheath includes a fluid chamber that when inflated expands the sheath from an unexpanded configuration in which the central lumen has a first diameter to an expanded configuration in which the central lumen has a second, larger, diameter.

### CROSS-REFERENCE TO RELATED APPLICATIONS

This application is a continuation of International Application No. PCT/US2024/016761, filed Feb. 21, 2024, which claims the benefit of U.S. Provisional Application No. 63/454,415, filed Mar. 24, 2023, the contents of which are incorporated herein by reference in its entirety.

### FILED

The present application is directed to a sheath for use with catheter-based technologies for repairing and/or replacing heart valves, as well as for delivering an implant, such as a prosthetic valve to a heart via the patient's vasculature.

### BACKGROUND OF THE INVENTION

Endovascular delivery catheter assemblies are used to implant prosthetic devices, such as a prosthetic valve, at locations inside the body that are not readily accessible by surgery or where access without invasive surgery is desirable. For example, aortic, mitral, tricuspid, and/or pulmonary prosthetic valves can be delivered to a treatment site using minimally invasive surgical techniques.

Percutaneous interventional medical procedures utilize the large blood vessels of the body reach target destinations rather than surgically opening target site. There are many types of diseases states that can be treated via interventional methods including coronary blockages, valve replacements (TAVR) and brain aneurysms. These techniques involve using wires, catheters, balloons, electrodes and other thin devices to travel down the length of the blood vessels from the access site to the target site. The devices have a proximal end which the clinician controls outside of the body and a distal end inside the body which is responsible for treating the disease state. Percutaneous interventional procedures offer several advantages over open surgical techniques. First, they require smaller incision sites which reduces scarring and bleeding as well as infection risk. Procedures are also less traumatic to the tissue, so recovery times are reduced. Finally, interventional techniques can usually be performed much faster, and with fewer clinicians participating in the procedure, so overall costs are lowered. In some cases, the need for anesthesia is also eliminated, further speeding up the recovery process and reducing risk.

A single procedure typically uses several different guidewires, catheters, and balloons to achieve the desired effect. One at a time, each tool is inserted and then removed from the access site sequentially. For example, a guidewire is used to track to the correct location within the body. Next a balloon may be used to dilate a section of narrowed blood vessel. Last, an implant may be delivered to the target site. Because catheters are frequently inserted and removed, introducer sheaths are used to protect the local anatomy and simplify the procedure.

An introducer sheath can be used to safely introduce a delivery apparatus into a patient's vasculature (for example, the femoral artery). Introducer sheaths are conduits that seal onto the access site blood vessel to reduce bleeding and trauma to the vessel caused by catheters with rough edges. An introducer sheath generally has an elongated sleeve that is inserted into the vasculature and a housing that contains one or more sealing valves that allow a delivery apparatus to be placed in fluid communication with the vasculature with minimal blood loss. Once the introducer sheath is positioned within the vasculature, the shaft of the delivery apparatus is advanced through the sheath and into the vasculature, carrying the prosthetic device. Expandable introducer sheaths, formed of highly elastomeric materials, allow for the dilating of the vessel to be performed by the passing prosthetic device. However, portions of the sheath resist expansion requiring higher push forces for advancement of the delivery device and implant to the treatment location. Accordingly, there remains a need for systems and methods that reduce the push force needed to introduce the delivery device and implant to a treatment location within a patient's blood vessel.

### SUMMARY OF THE INVENTION

Aspects of the present expandable sheath system can minimize trauma to the vessel and damage to the sheath and prosthetic device by reducing push forces through the sheath. Some examples ensure that the sheath is not damaged in an effort to dilate or expand the strain relief portion. Some examples can comprise a sheath with a smaller profile than that of prior art introducer sheaths. Furthermore, certain examples can reduce the length of time a procedure takes, as well as reduce the risk of a longitudinal or radial vessel tear, or plaque dislodgement because lower push force is required and only one sheath is used, rather than several different sizes of sheaths.

In one of its basic configurations, the present disclosure provides an inflatable sheath for delivering a medical device. This basic configuration can preferably be provided with any one or more of the features described elsewhere herein, in particular with those of the examples described hereafter. However, it should be understood that the basic configuration can preferably also be provided with any one or more of the features shown in the figures and/or described in conjunction with the figures, either in addition to or alternatively to the features of the examples described hereafter.

In some examples, the sheath includes a fluid chamber (for example, a fluid-bearing chamber) that when inflated expands the sheath from an unexpanded configuration in which the central lumen has a first diameter to an expanded configuration in which the central lumen has a second, larger, diameter.

In some examples, the sheath includes: an inner layer defining the central lumen extending therethrough; an outer layer provided over the inner layer such that the fluid chamber is defined therebetween; and an inflation port in fluid communication with the fluid chamber.

In some examples, upon inflation of the fluid chamber, at least a portion of the sheath (for example, inner layer and/or outer layer) expands from an unexpanded configuration in which the central lumen has a first diameter to an expanded configuration in which the central lumen has a second, larger, diameter.

In some examples, upon deflation of the fluid chamber (for example, upon removal of the inflation fluid), at least a portion of the sheath (for example, inner layer and/or outer layer) returns toward the unexpanded configuration.

In some examples, in the expanded configuration, the inner and outer layers each expand from a first diameter to a second, larger, diameter (for example, the expanded outer diameter of the outer layer ranging from 14 French to 22 French, for example, 16 French).

In some examples, in the expanded configuration, the inner and outer layers increase at the same ratio (for example, the inner and outer layers experience the same percentage of increase in diameter compared to their corresponding unexpanded diameters).

In some examples, in the expanded configuration, the inner and outer layers increase at different ratios (for example, the inner and outer layers experience a different percentage of increase in diameter compared to their corresponding unexpanded diameters).

In some examples, in the expanded configuration, the outer layer increases at a greater ratio than the inner layer (for example, the diameter of the outer layer increased by a greater percentage than the diameter of the inner layer).

In some examples, in the expanded configuration, the inner layer increases at a greater ratio than the outer layer (for example, the diameter of the inner layer increased by a greater percentage than the diameter of the outer layer).

In some examples, the inner layer is composed of a different material than the outer layer such that in the expanded configuration, the inner and outer layers increase at different ratios.

In some examples, the outer layer is composed of a more elastic material than the inner layer such that the outer layer increases at a greater ratio than the inner layer.

In some examples, the inner layer is composed of a more elastic material than the outer layer such that the inner layer increases at a greater ratio than the outer layer.

In some examples, in the unexpanded configuration, the fluid chamber has a first volume and in the expanded configuration the fluid chamber has a second, larger, volume.

In some examples, the sheath further comprises an inflation fluid (for example, a fluid (saline), a gas) provided to the fluid chamber via the inflation port (the inflation port in fluid communication with a fluid port provided on the sheath hub), wherein the sheath expands from the unexpanded configuration to the expanded configuration upon receipt of the inflation fluid in the fluid chamber (for example, the sheath expands in response to the outwardly directed radial force exerted on the inner and/or outer layer from the increased volume of the inflation fluid received within the fluid chamber), and wherein the sheath moves from the expanded configuration toward the unexpanded configuration upon withdrawal of the inflation fluid from the fluid chamber (for example, the sheath returns toward the unexpanded configuration in response a reduction in the outwardly directed radial force exerted on the inner and/or outer layer by the inflation fluid resulting from a decrease in the volume of the inflation fluid received within the fluid chamber).

In some examples, the sheath further comprises an inflation fluid provided to the fluid chamber via the inflation port, wherein, in the unexpanded configuration, the fluid chamber receives a first volume of the inflation fluid, and in the expanded configuration the fluid chamber receives a second, larger, volume of the inflation fluid.

In some examples, the inner layer and the outer layer are composed of an elastomeric material (e.g., silicon, polyimide, polytetrafluoroethylene (PTFE), expanded polytetrafluorocthylene (ePTFE), ethylene tetrafluoroethylene (ETFE), nylon, polyethylene, polyamide, polyether block amide (e.g., Pebax), and/or combinations thereof), where a stiffness or elasticity of the sheath in the expanded (and/or unexpanded) configuration is constant along a length of the sheath (for example, constant along the inner and outer layer).

In some examples, the inner layer and the outer layer are composed of an elastomeric material (for example, silicon, polyimide, polytetrafluoroethylene (PTFE), expanded polytetrafluoroethylene (ePTFE), ethylene tetrafluoroethylene (ETFE), nylon, polyethylene, polyamide, polyether block amide (for example, Pebax), and/or combinations thereof), where a stiffness or elasticity of the sheath in the expanded (and/or unexpanded) configuration varies along a length of the sheath.

In some examples, the inner layer includes a plurality of segments of varying stiffness or elasticity, the plurality of segments including a first segment having a first stiffness (or a first elasticity) and a second segment having a second, different, stiffness (or a second lesser elasticity).

In some examples, at least one of the plurality of segments of the inner layer is composed of a different material than an other one of the plurality of segments of the inner layer having a different stiffness or elasticity (for example, first and second segments of the inner layer are composed of different materials of varying stiffness or elasticity).

In some examples, the outer layer includes a plurality of segments of varying stiffness or elasticity, the plurality of segments including a first segment having a first stiffness (or a first elasticity) and a second segment having a second, different, stiffness (or a second lesser elasticity).

In some examples, at least one of the plurality of segments of the outer layer is composed of a different material than an other one of the plurality of segments of the outer layer having a different stiffness or elasticity (for example, first and second segments of the outer layer are composed of different materials of varying stiffness or elasticity).

In some examples, the sheath includes a plurality tubular segments (for example, first tubular segment, second tubular segment, the number of tubular segments ranging from 1 to 10) defining corresponding plurality of fluid chambers (for example, first fluid chamber, second fluid chamber) between the inner and outer layers.

In some examples, the corresponding inner and outer layers of each of the plurality of tubular segments is composed of materials having different stiffness or elasticity (alternatively, the inner and outer layers of each of the plurality of tubular segments are composed of the same elastomeric material).

In some examples, the inflation port is in fluid communication with the fluid chambers of each of the plurality of tubular segments.

In some examples, each of the plurality of tubular segments is separately connected to the inflation port.

In some examples, each of the plurality of tubular segments is separately connected to a corresponding one of a plurality of inflation ports.

In some examples, each of the tubular segments is fluidly connected to the inflation port by an inflation lumen (for example, an inflation lumen passing between the inner and outer layers).

In some examples, each of the tubular segments is fluidly connected to a respective inflation port by a corresponding inflation lumen (for example, a plurality of inflation lumens passing between the inner and outer layers).

In some examples, in the unexpanded configuration, the fluid chamber of each of the corresponding tubular segments has a first volume, and in the expanded configuration, the fluid chamber of each of the corresponding tubular segments has a second, larger, volume.

In some examples, the volume of each of the plurality of tubular segments varies from at least one of an other tubular segment (for example, wherein, in the expanded configuration, the first tubular segment receives a first volume of inflation fluid, and the second tubular segment receives a second, larger, volume of inflation fluid).

In some examples, in the expanded configuration, the stiffness of each of the plurality of tubular segments varies in relation to their corresponding volume (for example, a tubular segment having greater volume of inflation fluid is stiffer than a tubular segment having a smaller volume of inflation fluid).

In some examples, the sheath further comprises an inflation fluid provided to the fluid chamber via the inflation port, wherein the volume of inflation fluid introduced into and/or withdrawn from each of the plurality of tubular segments is separately controlled.

In some examples, the volume of inflation fluid introduced into each of the plurality of tubular segments is separately controlled to vary the expansion of the tubular segments (for example, the outer layer and/or inner layer/central lumen).

In some examples, the volume of inflation fluid introduced into each of the plurality of tubular segments is controlled to incrementally expand the sheath (for example, incrementally expand the inner and outer layers along a length of the sheath).

In some examples, the volume of inflation fluid introduced into and/or withdrawn from each of the plurality of tubular segments is separately controlled by a processor.

In some examples, in the expanded configuration, at least a portion of the sheath is configured to locally expand from second diameter to a third, larger, diameter (for example, due to an outwardly directed radial force exerted on the central lumen of the inner layer by a medical device against the inner layer), and then locally contract at least partially back to the second diameter.

In some examples, the sheath includes a tubular strain relief layer provided over the inner layer positioned at a proximal end of the sheath and extending along at least a portion of a length of the sheath, wherein the strain relief layer comprises a stiffer and/or less elastomeric material than the inner layer and/or outer layers that restricts expansion of the inner and outer layers.

In some examples, strain relief layer includes: a proximal portion adjacent a proximal end of the strain relief layer; a distal portion adjacent a distal end of the strain relief layer; and a tapered portion extending between the distal portion and the proximal portion, wherein a diameter of the proximal portion is greater than a diameter of the distal portion.

In some examples, at least a portion of the strain relief layer is configured to locally expand from an unexpanded configuration at a first diameter to an expanded configuration at a second diameter (for example, due to an outwardly directed radial force exerted on the lumen of the inner layer by a medical device against the inner layer), and then locally contract at least partially back to the unexpanded configuration (for example, as the medical device passes through the lumen).

In some examples, the strain relief layer comprises a material having a higher durometer than at least one of the inner layer and/or the outer layer.

In some examples, the strain relief layer comprises polyurethane (for example, high density polyethylene).

In some examples, the sheath further includes an outer introducer sheath provided over the outer layer, the outer introducer sheath movable along the outer layer, outer introducer sheath comprising: a continuous first layer (inner layer) defining a lumen extending therethrough, the inner layer having at least one folded portion; and a second layer provided over the first layer, where the second layer is discontinuous and includes an overlapping portion and an underlying portion, and the overlapping portion overlaps the underlying portion, wherein the outer introducer sheath is configured to locally expand from an unexpanded configuration in the which the lumen has a first diameter to an expanded configuration in which the lumen has a second diameter that is larger than the first diameter (for example, due to an outwardly directed radial force exerted on the lumen of the inner layer by the inflation of the fluid chambers and expansion of the inner and outer layers), and then locally contract at least partially back to the unexpanded configuration.

In some examples, when in the unexpanded configuration, the folded portion extends circumferentially over an outer surface of the inner layer and/or outer layer, wherein at least a portion of the folded portion of the inner layer is positioned between the overlapping and underlying portions.

In some examples, in the expanded configuration local expansion causes a length of the folded portion to at least partially unfold, wherein in the expanded configuration local expansion of the sheath causes a length of the overlapping portion to move circumferentially with respect to the underlying portion, wherein in the expanded configuration, local expansion of the sheath forms a gap between longitudinally extending edges of the outer layer, wherein at least a portion of the unfolded portion extends into the gap.

In some examples, the sheath further includes an elastic outer jacket (for example, PEBAX, polyurethane, silicone, or polyisoprene, or combination thereof) extending at least partially over the sheath (for example, at least partially over the inner layer, the outer layer, and/or the strain relief layer) where the outer cover locally expands and contracts as the sheath moves between the expanded and unexpanded configurations, wherein the elastic outer cover exerts a radially inward force on the sheath (for example, urging the inner layer, outer layer, and or strain relief layer toward the unexpanded configuration).

A further example of the present disclosure provides a method delivering a medical device through a sheath including: providing the sheath including an inner layer defining a central lumen extending therethrough and an outer layer provided over the inner layer such that a fluid chamber is defined therebetween; introducing an inflation fluid into the fluid chamber by an inflation port in fluid communication with the fluid chamber thereby expanding at least a portion of the sheath (for example, inner layer and/or outer layer) from an unexpanded configuration in which the central lumen has a first diameter to an expanded configuration in which the central lumen has a second, larger, diameter; and withdrawing the inflation fluid from the fluid chamber by the inflation port thereby moving at least a portion the sheath (for example, inner layer and/or outer layer) toward the unexpanded configuration.

In some examples, the sheath expands from the unexpanded configuration to the expanded configuration upon receipt of the inflation fluid in the fluid chamber (for example, the sheath expands in response to the outwardly directed radial force exerted on the inner and/or outer layer in response to the increased volume of the inflation fluid received within the fluid chamber), wherein the sheath moves from the expanded configuration toward the unexpanded configuration upon withdrawal of the inflation fluid from the fluid chamber (for example, the sheath returns toward the unexpanded configuration in response a reduction in the outwardly directed radial force exerted on the inner and/or outer layer by the inflation fluid resulting from a decrease in the volume of the inflation fluid received within the fluid chamber).

In some examples, the sheath includes a plurality of tubular segments defining corresponding plurality of fluid chambers (for example, first fluid chamber, second fluid chamber) between the inner and outer layers, and introducing an inflation fluid into the fluid chamber further includes: varying the volume of the inflation fluid introduced into of the plurality of tubular segments, such that a volume of the inflation fluid introduced into one of the plurality of tubular segments varies from at least one of an other tubular segment, wherein, in the expanded configuration, the stiffness of each of the plurality of tubular segments varies in relation to their corresponding volume of inflation fluid.

In some examples, the volume of inflation fluid introduced into and/or withdrawn from each of the plurality of tubular segments is separately controlled.

In some examples, the volume of inflation fluid introduced into each of the plurality of tubular segments is controlled to vary the expansion of the tubular segments (for example, the outer layer and/or inner layer/central lumen).

In some examples, the volume of inflation fluid introduced into each of the plurality of tubular segments is controlled to incrementally expand the sheath (for example, incrementally expand the inner and outer layers along a length of the sheath).

Another implementation of the present disclosure provides a method of delivering a medical device through a sheath, the method including: providing the sheath including an inner layer defining a central lumen extending therethrough and an outer layer provided over the inner layer such that a fluid chamber is defined therebetween; introducing an inflation fluid into the fluid chamber by an inflation port in fluid communication with the fluid chamber thereby expanding at least a portion of the sheath (for example, inner layer and/or outer layer) from an unexpanded configuration in which the central lumen has a first diameter to an expanded configuration in which the central lumen has a second, larger, diameter; introducing a medical device into the central lumen of the sheath; advancing the medical device through the central lumen of the sheath; and withdrawing the medical device from the central lumen of the sheath; and withdrawing the inflation fluid from the fluid chamber by the inflation port thereby moving at least a portion the sheath (for example, inner layer and/or outer layer) toward the unexpanded configuration.

In some examples, sheath includes a plurality of tubular segments defining corresponding plurality of fluid chambers (for example, first fluid chamber, second fluid chamber) between the inner and outer layers, and introducing an inflation fluid into the fluid chamber further includes: varying the volume of the inflation fluid introduced into of the plurality of tubular segments, such that a volume of the inflation fluid introduced into one of the plurality of tubular segments varies from at least one of an other tubular segment, wherein, in the expanded configuration, the stiffness of each of the plurality of tubular segments varies in relation to their corresponding volume of inflation fluid.

A further implementation of the present disclosure provides a method of inserting a medical device into a blood vessel of a patient, the method including: providing the sheath including an inner layer defining a central lumen extending therethrough and an outer layer provided over the inner layer such that a fluid chamber is defined therebetween; inserting the sheath at least partially into the blood vessel of the patient; introducing an inflation fluid into the fluid chamber by an inflation port in fluid communication with the fluid chamber thereby expanding at least a portion of the sheath (for example, inner layer and/or outer layer) from an unexpanded configuration in which the central lumen has a first diameter to an expanded configuration in which the central lumen has a second, larger, diameter; introducing the medical device into the central lumen of the sheath; advancing the medical device through the central lumen of the sheath; advancing the medical device beyond a distal opening in the sheath to a treatment site within the blood vessel; withdrawing the medical device from the central lumen of the sheath; withdrawing the inflation fluid from the fluid chamber by the inflation port thereby moving at least a portion the sheath (for example, inner layer and/or outer layer) toward the unexpanded configuration; and removing the sheath from the blood vessel of the patient.

In some examples, the sheath expands from the unexpanded configuration to the expanded configuration upon receipt of the inflation fluid in the fluid chamber (for example, the sheath expands in response to the outwardly directed radial force exerted on the inner and/or outer layer in response to the increased volume of the inflation fluid received within the fluid chamber).

In some examples, the sheath moves from the expanded configuration toward the unexpanded configuration upon withdrawal of the inflation fluid from the fluid chamber (for example, the sheath returns toward the unexpanded configuration in response a reduction in the outwardly directed radial force exerted on the inner and/or outer layer by the inflation fluid resulting from a decrease in the volume of the inflation fluid received within the fluid chamber).

In some examples, the sheath includes a plurality of tubular segments defining corresponding plurality of fluid chambers (for example, first fluid chamber, second fluid chamber) between the inner and outer layers), wherein introducing an inflation fluid into the fluid chamber further includes: varying the volume of the inflation fluid introduced into of the plurality of tubular segments, such that a volume of the inflation fluid introduced into one of the plurality of tubular segments varies from at least one of an other tubular segment, wherein, in the expanded configuration, the stiffness of each of the plurality of tubular segments varies in relation to their corresponding volume of inflation fluid the method includes

In some examples, a first volume of inflation fluid is introduced into the fluid chamber before inserting the sheath at least partially into the blood vessel of the patient the method includes, wherein the first volume of the inflation fluid increases a volume of the fluid chamber, wherein at least a portion of the sheath is partially expanded the unexpanded configuration toward the expanded configuration increasing the stiffness of the sheath.

In some examples, advancing the medical device through the central lumen of the sheath further includes causing the sheath to locally expand from the unexpanded configuration to the expanded configuration at a location proximate the medical device in response to the outwardly directed radially force of the medical device exerted against the sheath (for example, inner layer), wherein withdrawing the medical device from the central lumen of the sheath further includes locally contracting the sheath at least partially back to the unexpanded configuration as the medical device (for example implant) passes through the central lumen.

In some examples, the method further includes withdrawing the medical device from the central lumen of the sheath; and withdrawing the inflation fluid from the fluid chamber by the inflation port thereby moving at least a portion the sheath (for example, inner layer and/or outer layer) toward the unexpanded configuration).

In some examples, the medical device is a prosthetic device mounted in a radially crimped state on a delivery apparatus, and the act of advancing the prosthetic device through the lumen of the sheath comprises advancing the delivery apparatus and the prosthetic device through lumen of the sheath and into the vasculature of the patient.

In some examples, the prosthetic device comprises a prosthetic heart valve and the method further comprises implanting the prosthetic heart valve at a treatment site within the patient.

In some examples, the prosthetic heart valve is mounted on a balloon catheter of the delivery apparatus as the prosthetic heart valve is advanced through the sheath.

In some examples, the sheath is inserted into a femoral artery of a patient.

Various aspects of the examples described herein can be combined based on desired sheath system characteristics.

### BRIEF DESCRIPTION OF DRAWINGS

FIG. 1 is an elevation view of an expandable sheath along with an endovascular delivery apparatus for implanting a prosthetic implant.

FIG. 2 is an elevation view of an expandable sheath including an introducer locking hub, a sheath locking sleeve, and an introducer.

FIG. 3 is an elevation view of the expandable sheath of FIG. 2 along with an endovascular delivery apparatus for implanting a prosthetic implant.

FIG. 4 is an elevation view of an expandable sheath a sheath hub, an introducer locking hub, and a sheath locking sleeve of FIG. 2.

FIG. 5A is a cross-sectional view of the sheath hub, introducer locking hub, and sheath locking sleeve of FIG. 2.

FIG. 5B is a cross-sectional view of the introducer cap, the sheath hub, the introducer locking hub, the sheath locking sleeve of FIG. 2.

FIG. 6 is a cross-sectional view of the introducer cap, sheath hub, introducer locking hub, and sheath locking sleeve of FIG. 2.

FIG. 7 is a distal end view of the sheath locking sleeve of FIG. 2 and the proximal fluid seal of FIGS. 5A-B.

FIG. 8A is a first elevation view of the introducer locking hub of FIG. 2 coupled to an introducer.

FIG. 8B is a second (rotated) elevation view of the introducer locking hub of FIG. 2 coupled to the introducer.

FIG. 8C is a distal end view of the introducer locking hub of FIG. 2 coupled to the introducer.

FIG. 8D is a partial side view of the introducer locking hub of FIG. 2 coupled to the introducer.

FIG. 8E is a partial perspective view of the introducer locking hub of FIG. 2 coupled to the introducer.

FIG. 8F is a partial perspective view of the introducer locking hub of FIG. 2 coupled to the introducer.

FIG. 9A is a distal end view of the introducer locking hub of FIG. 2.

FIG. 9B is a first elevation view of the introducer locking hub of FIG. 2.

FIG. 9C is a proximal end view of the introducer locking hub of FIG. 2.

FIG. 9D is a first perspective view of the introducer locking hub of FIG. 2.

FIG. 9E is a second elevation view of the introducer locking hub of FIG. 2.

FIG. 9F is a second perspective view of the introducer locking hub of FIG. 2.

FIG. 10A is a distal end view of the sheath locking sleeve of FIG. 2.

FIG. 10B is a first elevation view of the sheath locking sleeve of FIG. 2.

FIG. 10C is a proximal end view of the sheath locking sleeve of FIG. 2.

FIG. 10D is a first perspective view of the sheath locking sleeve of FIG. 2.

FIG. 10E is a second elevation view of the sheath locking sleeve of FIG. 2.

FIG. 10F is a second perspective view of the sheath locking sleeve of FIG. 2.

FIG. 11 is a side elevation cross-sectional view of a portion of the expandable sheath of FIGS. 1 and 2.

FIG. 12 is a magnified view of a portion of the expandable sheath of FIGS. 1 and 2.

FIG. 13A is a magnified view of a portion of the expandable sheath of FIGS. 1 and 2 with the outer layer removed for purposes of illustration.

FIG. 13B is a magnified view of a portion of the braided layer of the sheath of FIGS. 1 and 2.

FIG. 14 is a magnified view of a portion of the expandable sheath of FIGS. 1 and 2 illustrating expansion of the sheath as a prosthetic device is advanced through the sheath.

FIG. 15 is a side view of the expandable sheath of FIGS. 1 and 2.

FIG. 16 is a magnified cross-sectional view of the sheath of FIG. 15 along section line 16-16.

FIG. 17 is cross-sectional view of the unexpanded sheath of FIG. 16 along section line 17-17.

FIG. 18 is cross-sectional view of the unexpanded sheath of FIG. 15 along section line 18-18.

FIG. 19 is cross-sectional view of the unexpanded sheath of FIG. 15 along section line 19-19.

FIG. 20 is cross-sectional view of the expanded sheath of FIG. 15 along section line 19-19.

FIG. 21 is a side view of the expandable sheath of FIGS. 1 and 2.

FIG. 22 is a cross-sectional view of the unexpanded sheath of FIG. 21 along section line 22-22.

FIG. 23 is a cross-sectional view of the expanded sheath of FIG. 21 along section line 22-22.

FIG. 24 is a side view of the expandable sheath of FIGS. 1 and 2.

FIG. 25 is a partial cross-sectional view of the sheath of FIG. 24 along section line A-A in an unexpanded configuration.

FIG. 26 is a partial cross-sectional view of the sheath of FIG. 24 along section line A-A in an expanded configuration.

FIG. 27 is a cross-sectional view of the of the sheath of FIG. 24 along section line B-B in an unexpanded configuration.

FIG. 28 is a cross-sectional view of the of the sheath of FIG. 24 along section line B-B in an expanded configuration.

FIG. 29 is a partial cross-sectional view of the sheath of FIG. 24 along section line C-C.

FIG. 30 is a partial cross-sectional view of the sheath of FIG. 24 along section line C-C.

### DETAILED DESCRIPTION

The following description of certain examples of the inventive concepts should not be used to limit the scope of the claims. Other examples, features, aspects, implementations, and advantages will become apparent to those skilled in the art from the following description. As will be realized, the device and/or methods are capable of other different and obvious aspects, all without departing from the spirit of the inventive concepts. Accordingly, the drawings and descriptions should be regarded as illustrative in nature and not restrictive.

For purposes of this description, certain aspects, advantages, and novel features of the aspects of this disclosure are described herein. The described methods, systems, and apparatus should not be construed as limiting in any way. Instead, the present disclosure is directed toward all novel and nonobvious features and aspects of the various disclosed examples, alone and in various combinations and sub-combinations with one another. The disclosed methods, systems, and apparatus are not limited to any specific aspect, feature, or combination thereof, nor do the disclosed methods, systems, and apparatus require that any one or more specific advantages be present or problems be solved.

Features, integers, characteristics, compounds, chemical moieties, or groups described in conjunction with a particular aspect or example of the present disclosure are to be understood to be applicable to any other aspect or example described herein unless incompatible therewith. All of the features disclosed in this specification (including any accompanying claims, abstract, and drawings), and/or all of the steps of any method or process so disclosed, may be combined in any combination, except combinations where at least some of such features and/or steps are mutually exclusive. The present disclosure is not restricted to the details of any foregoing aspects. The present disclosure extends to any novel one, or any novel combination, of the features disclosed in this specification (including any accompanying claims, abstract, and drawings), or to any novel one, or any novel combination, of the steps of any method or process so disclosed.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

The terms “proximal” and “distal” as used herein refer to regions of a sheath, catheter, or delivery assembly. “Proximal” means that region closest to handle of the device, while “distal” means that region farthest away from the handle of the device.

“Axially” or “axial” as used herein refers to a direction along the longitudinal axis of the sheath.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal aspect. “Such as” is not used in a restrictive sense, but for explanatory purposes.

Disclosed examples of an expandable sheath can minimize trauma to the vessel by allowing for temporary expansion of a portion of the introducer sheath to accommodate the delivery system, followed by a return to the original diameter once the device passes through. Disclosed examples of the introducer sheath prevent the introducer from separating from the sheath during insertion by locking of the proximal hub of the introducer to the proximal hub of the sheath. Fixing the introducer and the sheath prevents the introducer from moving backward during insertion, thereby maintaining a snug fit and smooth transition between the introducer and the distal end of the sheath. Furthermore, present examples can reduce the length of time a procedure takes, as well as reduce the risk of a longitudinal or radial vessel tear, or plaque dislodgement because only one sheath is required, rather than several different sizes of sheaths. Examples of the present expandable sheath can avoid the need for multiple insertions for the dilation of the vessel.

Disclosed herein are elongate introducer sheaths that are particularly suitable for delivery of implants in the form of implantable heart valves, such as balloon-expandable implantable heart valves. Balloon-expandable implantable heart valves are well-known and will not be described in detail here. An example of such an implantable heart valve is described in U.S. Pat. No. 5,411,552, and also in U.S. Pat. No. 9,393,110, both of which are hereby incorporated by reference. The expandable introducer sheaths disclosed herein may also be used to deliver other types of implantable medical device, such as self-expanding and mechanically expanding implantable heart valves, stents or filters. Beyond transcatheter heart valves, the introducer sheath system can be useful for other types of minimally invasive surgery, such as any surgery requiring introduction of an apparatus into a subject's vessel. For example, the introducer sheath system can be used to introduce other types of delivery apparatus for placing various types of intraluminal devices (for example, stents, stented grafts, balloon catheters for angioplasty procedures, etc.) into many types of vascular and non-vascular body lumens (for example, veins, arteries, esophagus, ducts of the biliary tree, intestine, urethra, fallopian tube, other endocrine or exocrine ducts, etc.). The term “implantable” as used herein is broadly defined to mean anything—prosthetic or not—that is delivered to a site within a body. A diagnostic device, for example, may be an implantable.

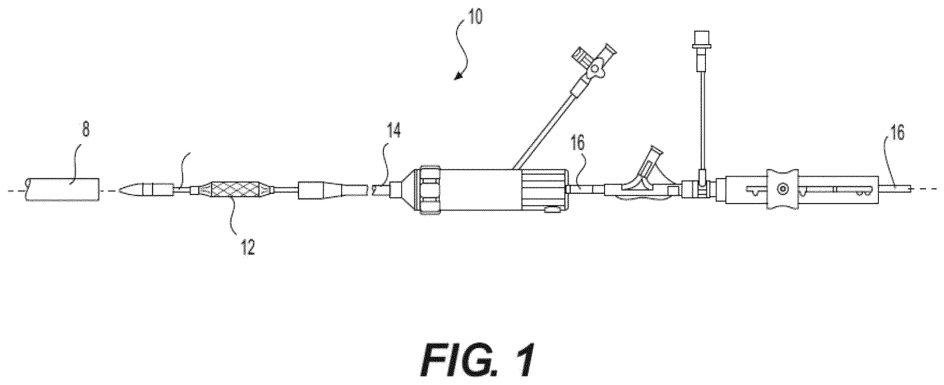

FIG. 1 illustrates an exemplary sheath 8 in use with a representative delivery apparatus 10, for delivering an implant 12, or other type of implantable (for example, tissue heart valve), to a patient. The delivery apparatus 10 can include a steerable guide catheter 14 (also referred to as a flex catheter) and a balloon catheter 16 extending through the guide catheter 14, and a nose catheter 15 extending through the balloon catheter 16. The guide catheter 14, balloon catheter 16, and nose catheter 15 in the illustrated example are adapted to slide longitudinally relative to each other to facilitate delivery and positioning of the implant 12 at an implantation site in a patient's body as described in detail herein. It is contemplated that the sheath 8 can be used with any type of elongated delivery apparatus used for implanting balloon-expandable prosthetic valves, self-expanding prosthetic valves, and other prosthetic devices.

As described in more detail herein, in general, the sheath 8 comprises an elongate expandable tube that, in use, is inserted into a vessel (for example, transfemoral vessel, femoral artery, iliac artery) by passing through the skin of patient, such that the distal end of the sheath 8 is inserted into the vessel. Sheath 8 includes a hemostasis valve and/or sealing features at the proximal end of the sheath, for example, in the sheath hub 20, that provide hemostasis and prevents blood leakage from the patient through the sheath 8. The sheath 8, including an introducer 6, is advanced into the patient's vasculature. Once positioned the introducer 6 is removed and the delivery apparatus 10 is inserted into/through the sheath 8, and the prosthetic device (implant 12) then be delivered and implanted within patient.

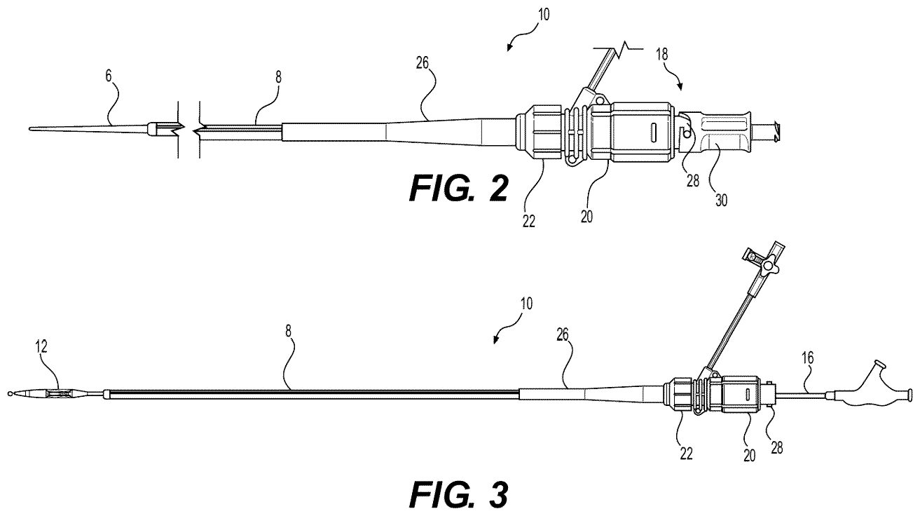

FIGS. 2 and 3, the introducer device/sheath assembly includes a sheath hub 20 at a proximal end of the device and an expandable sheath 8 extending distally from the sheath hub 20. The sheath 8 is coupled to the sheath hub 20 which in turn is removably coupled to a sheath locking system 18. The sheath locking system 18 allows the introducer 6, or other device desired to be removably couped (axially and rotatably) to the sheath 8.

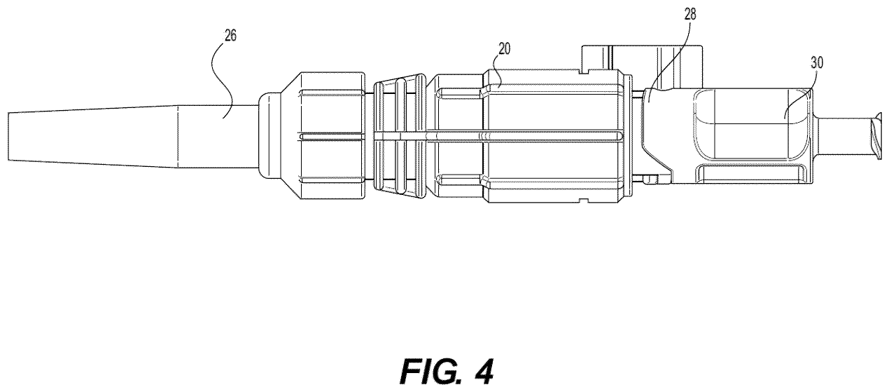

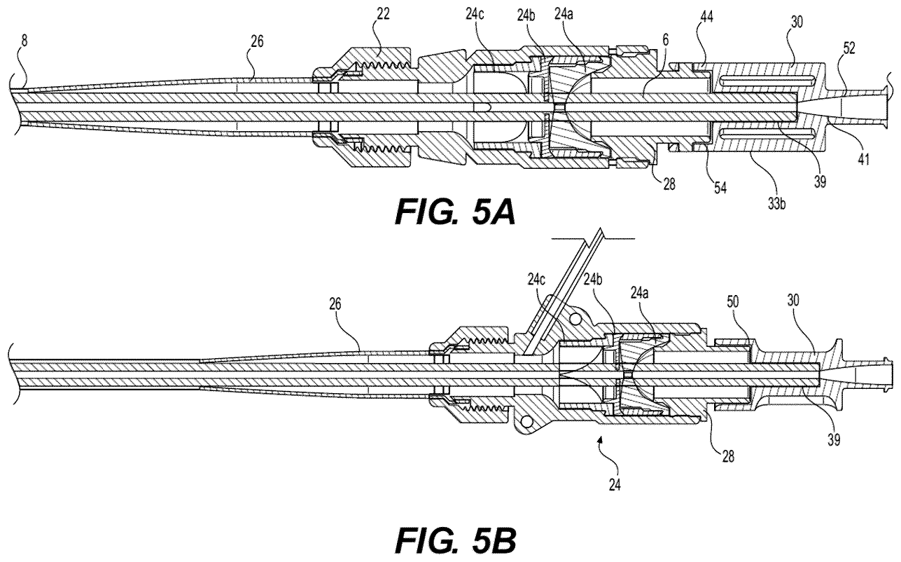

As illustrated in FIGS. 2-6, the sheath hub 20 can function as a handle for the device. Sheath hub 20 also provides a housing for necessary seal assemblies and an access point for a secondary lumen (for example, fluid lumen) in fluid communication with the central lumen of the sheath hub 20. The seal assembly 24, as described herein and as shown in FIGS. 5A and 5B, is included in the sheath hub 20. The seal assembly 24 includes a proximal seal 24a, an intermediate seal 24b, and a distal seal 24c. When assembled, the introducer 6 passes through the seal assembly and extends distal of the sheath 8. The proximal seal 24a, the intermediate seal 24b, and the distal seal 24c are each formed to prevent unwanted fluid from advancing in the proximal direction through the sheath hub 20 and proximal of the seal assembly 24. They are each openable and closable to provide pressure variation to affect the desired fluid flow from a physician or technician.

The distal end of the sheath hub 20 includes threads 21 for coupling to a threaded sheath hub cap 22. The sheath 8 is provided between the sheath hub 20 and the sheath hub cap 22 such that coupling the sheath hub cap 22 to the sheath hub 20 fixes the sheath 8 to the sheath hub 20. The sheath hub cap 22 is a cylindrical cap having a cap body having a proximal end and a distal end and defining a central lumen extending longitudinally between the proximal end and the distal end. The sheath hub cap 22 has a larger diameter at its proximal end than at its distal end.

The sheath hub 20 further has receiving slots 48 for coupling the sheath locking system 18, particularly the locking sleeve 28, to the sheath hub 20. The receiving slots 48 are openings which extend around a portion of the diameter of the sheath hub 20 and are sized and configured to accept the interference diameters 66 of the locking sleeve 28. Coupling between the receiving slots 48 and the interference diameters 66 axially and rotationally fixes the locking sleeve 28 and the sheath hub 20 relative to each other.

FIG. 2 illustrates the sheath 8 of FIG. 1 including a sheath locking system 18 which prevents axial and rotational translation of the introducer 6 with respect to the sheath 8. Example locking systems are disclosed in PCT/US2021/050006, entitled “Expandable Sheath Including Reverse Bayonet Locking Hub,” the disclosure of which is incorporated herein by reference. It is contemplated that the locking system disclosed herein can also be used to couple the sheath 8/sheath hub 20 with other delivery system components, catheters, dilators, etc. including the same mating features.

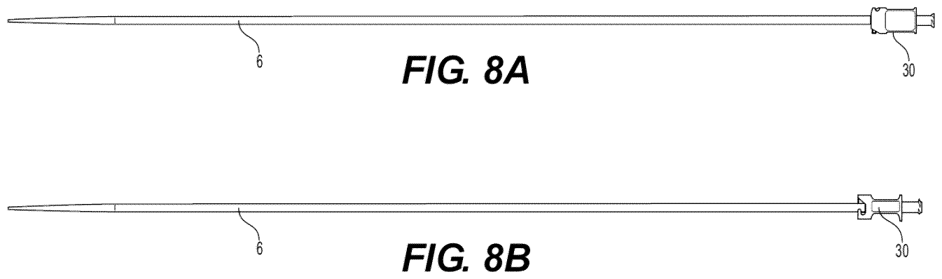

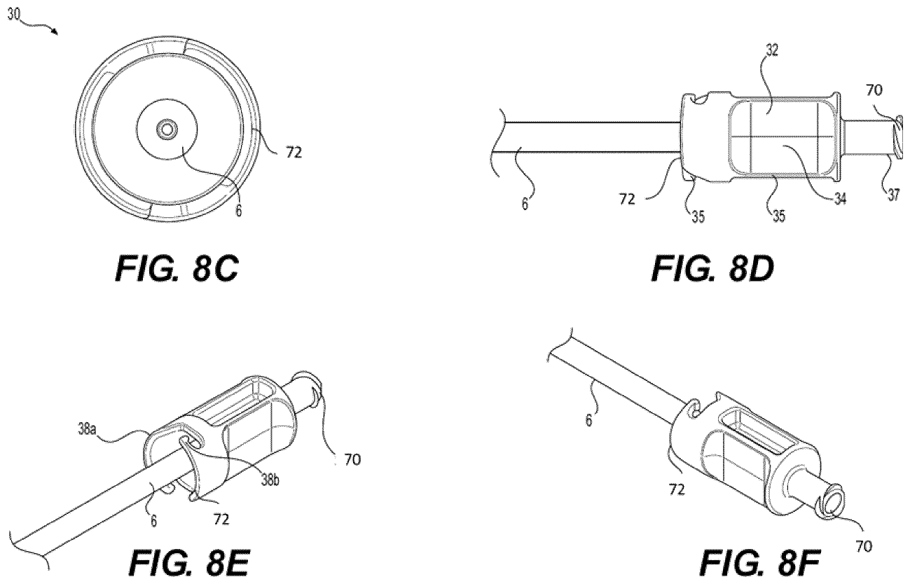

The sheath locking system 18 keeps the introducer 6 fixed with respect to the sheath 8 during insertion without requiring a physician or technician to hold the introducer 6 and the sheath 8 in place at the distal end. As illustrated in FIGS. 8A-8B, the sheath locking system 18 includes a locking sleeve 28 and an introducer locking hub 30 (including corresponding introducer 6). The locking sleeve 28 is coupled to the sheath 8 via the sheath hub 20. The locking sleeve 28 engages the introducer locking hub 30 and is moveable between a locked and unlocked position, thereby fixing the position of the introducer 6 and the sheath 8 and preventing movement therebetween, particularly during insertion into the patient. As will be described in more detail herein, the sheath locking system 18 keeps the introducer 6 from separating from the sheath 8 and prevents gaps from forming that can cause patient abrasions and unintended fluid flow between the introducer 6 and the sheath 8 during insertion.

FIGS. 2, 5A-5B and 6, and illustrate the sheath locking sleeve 28 coupled to the introducer locking hub 30 and the sheath hub 20. As will be described in more detail herein, the locking sleeve 28 includes a guide 31 that engages a locking channel 38 provided on the introducer locking hub 30. The guide 31 moves within the locking channel 38 between an unlocked position, where the sheath locking sleeve 28 is rotationally and axially movable with respect to the introducer locking hub 30, and a locked position (FIG. 2), where the locking sleeve 28 is axially fixed with respect to the introducer locking hub 30.

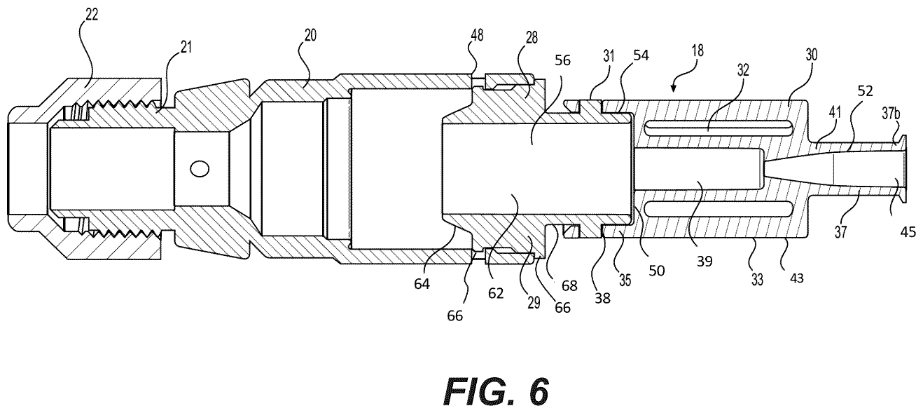

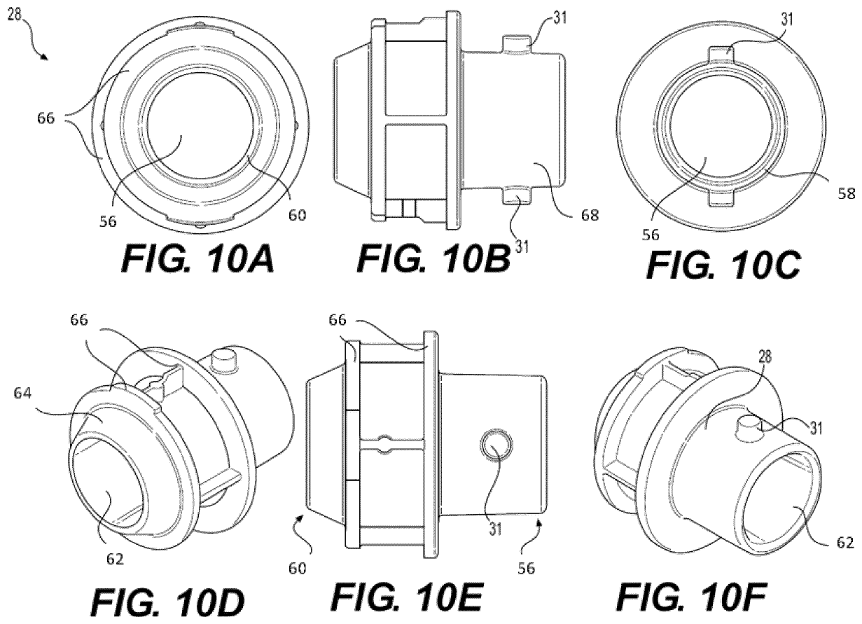

The locking sleeve 28 is illustrated, for example, in FIGS. 10A-10F. The locking sleeve 28 includes an elongated sleeve body 29 with a central lumen 56 extending longitudinally between the proximal end 58 and distal end 60 of the sleeve body 29. As provided in FIG. 6, the central lumen 56 defines a generally cylindrical inner surface 62 of the sheath locking sleeve 28. The central lumen 56 has a diameter of at least 0.3″. In some examples, the diameter ranges between 0.3″ and 0.6″. Preferably, the diameter is about 0.40″. The distal end 60 of the sleeve body 29 also has a frustoconical outer surface 64 that tapers about the distal end 60 to help with positioning the locking sleeve 28 within the sheath hub 20 and abutting the seal assembly 24 (FIGS. 5B and 5B). The locking sleeve 28 also has a plurality of interference diameters 66 that extend radially from the outer surface of the sleeve body 29 around (all or a portion of) the circumference of the locking sleeve 28. As illustrated in FIGS. 5A and 6, the distal interference diameters 66 are sized and configured to engage corresponding recesses and/or receiving slots 48 provided in the sheath hub 20 for securing the locking sleeve 28 to the sheath hub 20, and the distal interference diameter 66 seat against the proximal end of the sheath hub 20.

The locking sleeve 28 includes a guide 31 projecting from the outer surface 68 of the locking sleeve 28. The guide 31 engages a correspondingly-shaped locking channel 38 in the introducer locking hub 30. The guide 31 extends radially from the outer surface 68 and at least partially around the circumference of the outer surface 68. As provided in FIG. 6, the top surface of the guide 31 does not extend beyond the outer surface of the introducer locking hub 30 when the sheath locking sleeve 28 and the introducer locking hub 30 are coupled. For example, the height of the guide 31 corresponds to the wall thickness of the introducer locking hub 30 proximate the guide when the sheath locking sleeve 28 and the introducer locking hub 30 are coupled. In another example, the top surface of the guide 31 is recessed with respect to the outer surface of the introducer locking hub 30. That is, the height of the guide 31 is less than the wall thickness of the introducer locking hub 30. In some examples, the height of the guide 31 is greater than a wall thickness of the introducer locking hub 30 such that the top surface of the guide 31 extends beyond the outer surface of the introducer locking hub 30 when the sheath locking sleeve 28 and the introducer locking hub 30 are coupled. In some examples, the height/axial length of the guide 31 is between about 0.050″ and about 0.10.″ In some examples that height/axial length of the guide 31 is about 0.075″.

As illustrated in FIGS. 10D-10F, the guide 31 is a cylindrically-shaped projection. However, it is contemplated that the guide 31 may have any other regular or irregular shape that would facilitate movement of the guide 31 within the locking channel 38 of the introducer locking hub 30. For example, the guide 31 may have an elongated hexagon shape. The guide 31 can have a diameter/width ranging from about 0.05″ to about 0.20″. Preferably the guide 31 has a diameter/width of about 0.100″.

In general, the locking sleeve 28 can be formed from polycarbonate, but in some examples, the locking sleeve 28 can be formed from rigid plastic, or any other material suitable for providing a strong locking connector for an introducer 6 (metal, composite, etc.).

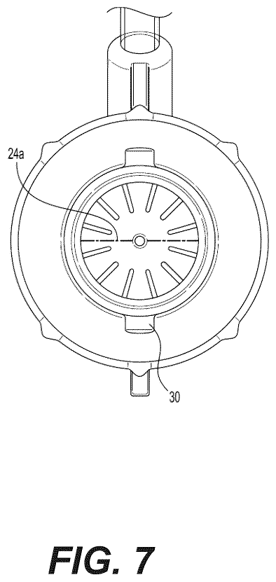

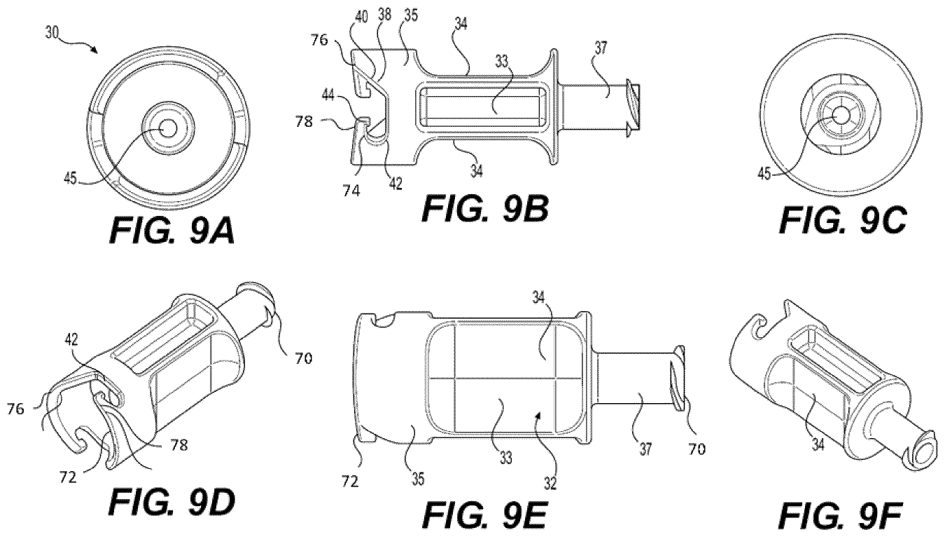

FIGS. 2-7 illustrate the introducer locking hub 30 coupled to the locking sleeve 28. FIGS. 8A-8F show the introducer locking hub 30 coupled to the introducer 6. FIGS. 9A-9F provide multiple view of the introducer locking hub 30. As described herein, the introducer 6 is fixedly coupled to the introducer locking hub 30. The introducer locking hub 30 couples with the locking sleeve 28 to fix the position the introducer 6 (axially and rotationally) with respect to the locking sleeve 28/sheath 8. Each of the introducer 6 and introducer locking hub 30 are described in more detail as follows.

FIGS. 8A-8F illustrate the introducer locking hub 30 with the introducer 6 coupled thereto. Example introducer sheaths are described, for example in U.S. Pat. Nos. 8,690,936 and 8,790,387, the disclosures of which are incorporated herein by reference. As provided in the cross-section views of FIGS. 5A and 5B, the introducer 6 is coupled to the introducer locking hub 30 and extends beyond the distal end of the introducer locking hub 30 body and into the sheath 8. When coupled to the sheath hub 20, the introducer 6 extends through the central lumen 56 of the sheath locking sleeve 28, the sheath hub 20 and the central lumen of the sheath 8. As will be descried herein, the sheath 8 generally comprises a radially expandable tubular structure. Passage of the introducer 6 through the sheath 8 and into a patient's vasculature causes the blood vessel to radially expand to about the diameter of the sheath 8. That is, the diameter of the central lumen of the sheath 8 is generally abuts the outer diameter of the introducer 6 such that the introducer 6 provides a mechanism to expand a patient's vessel to accept the sheath.

As provided in FIGS. 8A-8F, the introducer 6 is formed as an elongate body with a central lumen extending therethrough. As shown in FIGS. 5A and 5B, the central lumen of the introducer is aligned with the central lumens of the introducer locking hub 30, the sheath hub 20 and the sheath 8. The introducer 6 is received within a recessed opening 39 provided on an interior surface of the introducer locking hub 30, the recessed opening 39 axially aligned with the central lumen 45 of the introducer locking hub 30. The introducer 6 is coupled to the introducer locking hub 30 at the recessed opening 39. In an example system, the introducer 6 has a diameter corresponding to, or less than, the diameter of the recessed opening 39. In some examples, the introducer 6 is fixedly coupled to the introducer locking hub 30 at the recessed opening 39. For example, the introducer 6 is coupled to the recessed opening 39 of the introducer locking hub 30 by at least one of a press fit, an interference fit, a snap fit, a mechanical fastener, a chemical fastener (for example, an adhesive), a weld, a thermal process, and/or any other suitable coupling process known in the art.

As described herein, the introducer 6 has a central lumen that aligns with the central lumen 45 of the introducer locking hub 30. This joined lumen allows for the passage of surgical equipment and/or medical devices to the treatment site (for example, a guide wire). In an example system, and as provided in FIGS. 5A and 5B, the central lumen of the introducer 6 has a diameter corresponding to at least a portion of the diameter of the central lumen 45 of the introducer locking hub 30. In general, the corresponding diameter portion is adjacent the distal end of the central lumen 45. In some examples, the diameter of the central lumen 45 at the distal end of the introducer locking hub 30 is slightly larger than the diameter of the central lumen passing through the introducer 6. The central lumen 45 can also define a decreasing tapered portion 41 between the proximal end and the distal end of the introducer locking hub 30 (see FIG. 6). The corresponding diameter portion and decreasing tapered portion 41 allows for smooth transition and delivery of surgical equipment and/or medical device through the introducer locking hub 30 and into the central lumen of the introducer 6.

As illustrated in FIGS. 9A-9F, the introducer locking hub 30 includes a hub body 32 having a proximal end 70 and a distal end 72 and defining a central lumen 45 extending therethrough. The hub body 32 has a first (middle) portion 33, a second (distal) portion 35 which extends distally from the first portion 33 and a third (proximal) portion 37 which extends proximally from the first portion 33. The first portion 33 includes the cylindrically-shaped recessed opening 39 for receiving and retaining the introducer 6 and an outer surface 43. In some examples, the recessed opening 39 has a diameter ranging between 0.15″ and about 0.25″. In some examples, the recessed opening 39 has a diameter ranging between 0.17″ and about 0.20″. In some examples, the recessed opening has a diameter of about 0.194″.

The third (proximal) portion 37 of the introducer locking hub 30 includes the decreasing tapered portion 41 of the central lumen 45. The decreasing tapered portion 41 defining a frustoconical shape with decreasing taper/diameter from the proximal to the distal end of the sheath. It is contemplated that the tapered portion 41 has a minimum diameter of about 0.007″ and a maximum diameter of about 0.194″.

As illustrated in FIGS. 5A and B, when coupled, the central lumen 56 of the locking sleeve 28 is aligned with the central lumen 45 of the introducer locking hub 30. In some examples, the central lumen 56 of the locking sleeve 28 is coaxial with the central lumen 45 of the introducer locking hub 30. When coupled, the proximal end of the locking sleeve 28 is received within the central lumen 45 of the introducer locking hub 30. The proximal end surface of the locking sleeve 28 is adjacent a shoulder 50 provided on an inner surface of the central lumen 45 of the introducer locking hub 30. As illustrated in FIGS. 5A and 5B, the central lumen 45 of the introducer locking hub 30 includes a first portion 52 having a first diameter adjacent the proximal end of the introducer locking hub 30, and a second portion 54 having a second, larger, diameter adjacent the distal end of the introducer locking hub 30. The recessed opening 39 can be considered either a component of the first portion 52 of the central lumen 45, or a separate component of the central lumen 45 located between the first (proximal) portion 52 and the second (distal) portion 54. When the locking sleeve 28 and introducer locking hub 30 are coupled, at least a portion of the sleeve body 29 of the sheath locking sleeve 28 is received within the second portion 54 (larger portion) of the central lumen 45 of the introducer locking hub 30. The central lumen 56 of the sheath locking sleeve 28 is aligned with the central lumen 45 of the introducer locking hub 30 such that they are co-axial and form a smooth inner surface along the combined central lumens of the introducer locking hub 30 and the sheath locking sleeve 28.

As described generally herein, the locking sleeve 28 couples to the introducer locking hub 30 via engagement between the guide 31 on the locking sleeve 28 and the locking channel 38 provided in the introducer locking hub 30. As provided in FIGS. 9A-9F, the introducer locking hub 30 includes two locking channels 38. However, it is contemplated that the introducer locking hub 30 can include one locking channel 38 or more than two locking channels 38. The locking channel 38 can be is formed a recess or groove in a surface of the introducer locking hub 30, as a slotted opening, a clip, or as any other feature capable of receiving and securing the guide 31 projecting from the outer surface of the locking sleeve 28 with the introducer locking hub 30. Illustrated in FIG. 9B, the locking channels 38 provide an interface to secure the sheath locking sleeve 28 to the introducer locking hub 30 and ensure a fixed axial position between the introducer 6 and the sheath 8.

The locking channel 38 is formed on the distal end of the introducer locking hub 30. The locking channel 38 includes an opening on the distal end surface that leads to an angled guide portion 40 that transitions to a locking portion 42. The guide portion 40 is configured to direct the guide 31 of the locking sleeve 28 in an axial and circumferential direction along the side wall of the guide portion 40 towards the locking portion 42 upon rotation of the introducer locking hub 30 and/or the sheath locking sleeve 28. The locking portion 42 is configured to securely engage the guide 31, fixing the axial position of the introducer locking hub 30 with respect to the sheath locking sleeve 28. As illustrated in FIG. 9B, the guide portion 40 of the locking channel 38 extends from the distal end of the introducer locking hub 30 axially towards the proximal end of the introducer locking hub 30 and circumferentially around the introducer locking hub 30. For example, the guide portion 40 of the locking channel 38 can be described as extending helically around/along a length of the introducer locking hub 30 or on an angle from the distal end of the introducer locking hub 30.

As illustrated in FIGS. 9B and 9D, the locking portion 42 of the locking channel 38 extends at an angle from the end of the guide portion 40. As provided in FIG. 9B, the angle between the centerline of the guide portion 40 and the centerline of the locking portion 42 is greater than 90-degrees. In another example, the angle between the centerline of the guide portion 40 and the centerline of the locking portion 42 is about 120-degrees. In an example system, the locking portion 42 extends around a portion of the circumference of the introducer locking hub 30. The locking portion 42 can extend parallel to the distal end of the introducer locking hub 30. In an example system, the length of the guide portion 40 (measured along its centerline) is greater than a length of the locking portion 42 (measured along its centerline). In another example, the length of the guide portion 40 equals or is less than a length of the locking portion 42.

The locking portion 42 can include a catch 44 for securing the guide 31 within the locking portion 42 of the locking channel 38 and forming a partial barrier for the guide 31 within the locking portion 42. As illustrated in FIG. 9B, the catch 44 includes a projection that extends from a side wall 74 of the locking portion 42 and releasably secures the guide 31 within the locking channel 38. The catch 44 extends from the side wall 42a of the locking portion 42 in a proximal direction towards the center line of the locking portion 42 and has a height sufficient to retain the guide 31 between the catch 44 and the end of the locking portion 42.

The distal end surface of the introducer locking hub 30 can include features for biasing the guide 31 towards the locking channel 38. For example, the distal end of the introducer locking hub 30 can include a tapered surface angled toward an opening of the locking channel 38. As illustrated in FIG. 9B, the distal end 72 of the introducer locking hub 30 includes a first tapered surface 76 (angled towards a leading edge of the opening of the locking channel 38 and a second tapered surface 78 angled towards the trailing edge of the opening of the locking channel 38.

In use, engagement between the guide 31 and the guide portion 40 of the locking channel 38 is configured to bias the locking sleeve 28 in a proximal axial direction toward the proximal end 70 of the introducer locking hub 30 (towards a locked position) when the sheath locking sleeve 28 is rotated in a first axial direction. In this direction the guide 31 advances toward the locking portion 42 of the locking channel 38 into the locked position. Alternatively, engagement between the guide 31 and the locking portion 42 of the locking channel 38 is configured to bias the locking sleeve 28 in a distal axial direction toward the distal end of the introducer locking hub 30 (towards an unlocked position) when the sheath locking sleeve 28 is rotated in a second (opposite) axial direction. In the second direction, the guide 31 advances away from the locking portion 42 of the locking channel 38, to the unlocked position. When the guide 31 is in the locked position and retained with by locking portion 42 by catch 44, rotation in the second direction causes the guide 31 to bias against the catch 44 overcoming the oppositional forces of the catch 44, and moving the guide 31 from the locked to the unlocked position.

As illustrated in FIGS. 8A-9F, the outer surface of the introducer locking hub body 32 includes gripping features and/or surfaces for a physician or technician to use when manipulating the introducer locking hub 30. As provided in FIG. 9B, the introducer locking hub body 32 can include a two recessed gripping surfaces 34 on opposite sides of the longitudinal axis of the introducer locking hub 30. When the introducer locking hub 30 is viewed from the side, the gripping surfaces 34 define a dog-bone/barbell shape to the hub body 32, i.e., a shape having a smaller diameter/width center portion and larger diameter/width end portions. In an example system, the gripping surfaces 34 are provided along at least 40% of the length of the introducer locking hub body 32. In another example, the gripping surfaces 34 are provided along at least 50% of the length of the introducer locking hub body 32.

In general, the introducer locking hub 30 can be formed from polycarbonate, but in some examples the introducer locking hub 30 can be formed from rigid plastic, or any other material suitable for providing a locking mechanism for an introducer 6 (metal, composite, etc.).

As described herein, the introducer device/sheath assembly includes an expandable sheath 8 extending distally from the sheath hub 20. The expandable sheath 8 has a central lumen to guide passage of the delivery apparatus 10 for the medical device/prosthetic heart valve. In an alternative example, the introducer device/sheath assembly need not include the sheath hub 20. For example, the sheath 8 can be an integral part of a component of the sheath assembly, such as the guide catheter.

As described herein, the expandable sheath 8 can be formed from a highly elastomeric materials that allows for the dilating of the vessel to be performed by the passing prosthetic device. Example expandable introducer sheaths 8 are disclosed in U.S. Pat. No. 8,690,936, entitled “Expandable Sheath for Introducing an Endovascular Delivery Device into a Body,” U.S. Pat. No. 8,790,387, entitled “Expandable Sheath for Introducing an Endovascular Delivery Device into a Body,” U.S. Pat. No. 10,639,152, entitled “Expandable Sheath and Methods of Using the Same,” U.S. Pat. No. 10,792,471, entitled “Expandable Sheath,” U.S. patent application Ser. No. 16/407,057, entitled “Expandable Sheath with Elastomeric Cross Sectional Portions,” U.S. Pat. No. 10,327,896, entitled “Expandable Sheath with Elastomeric Cross Sectional Portions,” U.S. Pat. No. 11,273,062, entitled “Expandable Sheath,” Application No. PCT/US2021/019514, entitled “Expandable sheath for introducing an endovascular delivery device in to a body,” Application No. PCT/US2021/031227, entitled “Expandable sheath for introducing an endovascular delivery device into a body,” Application No. PCT/US2021/031275, entitled “Expandable sheath for introducing an endovascular delivery device into a body,” U.S. application Ser. No. 17/113,268, entitled “Expandable Sheath and Method of Using the Same,” Application No. PCT/US2021/058247, entitled “Self-Expanding, Two Component Sheath,” Application No. PCT/US2022/012785, entitled “Expandable Sheath,” U.S. Pat. No. 11,051,939, entitled “Active Introducer Sheath System,” Application No. PCT/US2022/012684, entitled “Introducer with Sheath Tip Expander,” U.S. application Ser. No. 17/078,556, entitled “Advanced Sheath Patterns,” Application No. PCT/US2021/025038, entitled “Low temperature hydrophilic adhesive for use in expandable sheath for introducing an endovascular delivery device into a body,” Application No. PCT/US2021/050006, entitled “Expandable Sheath Including Reversable Bayonet Locking Hub,” U.S. Provisional Application No. 63/280,251, entitled “Expandable Sheath Gasket to Provide Hemostasis,” the disclosures of which are herein incorporated by reference.

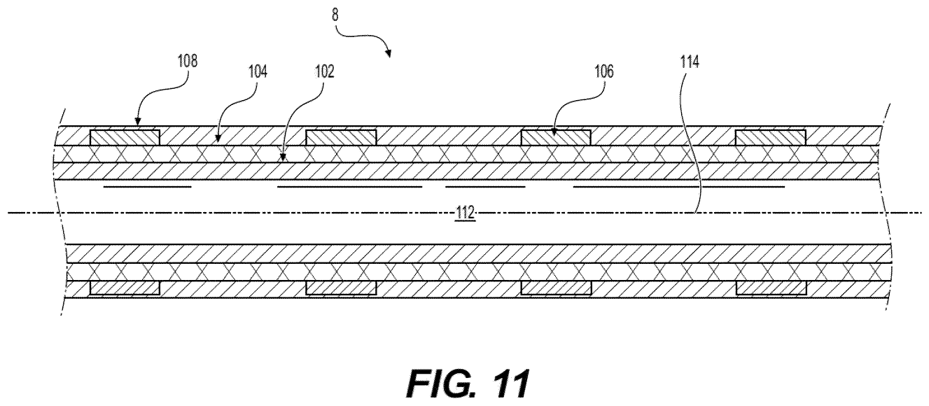

In certain examples, the expandable sheath 8 can comprise a plurality of coaxial layers extending along at least a portion of the length of the sheath 8. The structure of the coaxial layers is described in more detail herein with respect to FIGS. 11-23. Example expandable sheaths including coaxial layers are described, for example, in U.S. patent application Ser. No. 16/378,417, entitled “Expandable Sheath,” and U.S. patent application Ser. No. 17/716,882, entitled “Expandable Sheath,” the disclosures of which are herein incorporated by reference.

Various examples of the coaxial layered structure of the sheath 8 are described herein. For example, in reference to the example sheath 8 illustrated in FIGS. 11-14, the expandable sheath 8 can include a number of layers including an inner layer 102 (also referred to as an inner layer), a second layer 104 disposed around and radially outward of the inner layer 102, a third layer 106 disposed around and radially outward of the second layer 104, and a fourth outer layer 108 (also referred to as an outer layer) disposed around and radially outward of the third layer 106. In the illustrated configuration, the inner layer 102 can define the lumen 112 of the sheath extending along a central axis 114 through which the delivery apparatus travels into the patient's vessel in order to deliver, remove, repair, and/or replace a prosthetic device, moving in a direction along the longitudinal axis of the sheath 8.

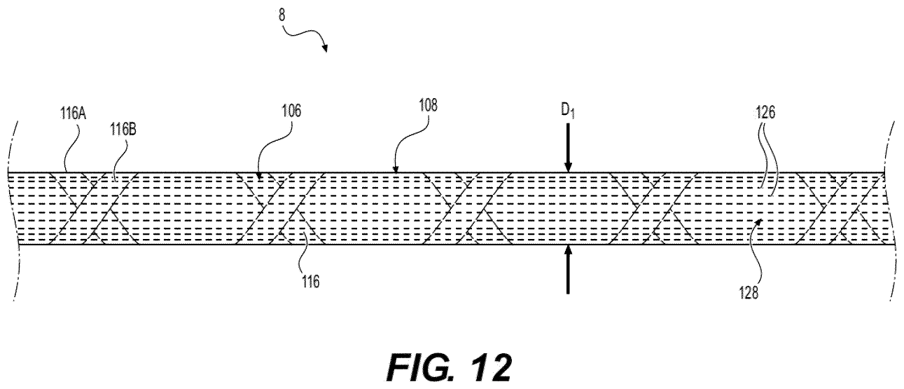

Referring to FIG. 12, when the sheath 8 is in an unexpanded state, various layers of the sheath, for example, the inner layer 102 and/or the outer layer 108, can form longitudinally-extending folds or creases such that the surface of the sheath comprises a plurality of ridges 126 (also referred to herein as “folds”). The ridges 126 can be circumferentially spaced apart from each other by longitudinally-extending valleys 128. When the sheath expands beyond its natural diameter D1, the ridges 126 and the valleys 128 can level out or be taken up as the surface radially expands and the circumference increases, as further described herein. When the sheath 8 collapses back to its natural diameter, the ridges 126 and valleys 128 can reform.

In certain examples, the inner layer 102 and/or the outer layer 108 can comprise a relatively thin layer of polymeric material. For example, the thickness of the inner layer 102 can be from 0.01 mm to 0.5 mm, 0.02 mm to 0.4 mm, or 0.03 mm to 0.25 mm. In some examples, the thickness of the outer layer 108 can be from 0.01 mm to 0.5 mm, 0.02 mm to 0.4 mm, or 0.03 mm to 0.25 mm.

In certain examples, the inner layer 102 and/or the outer layer 108 can comprise a lubricious, low-friction, and/or relatively non-elastic material. In particular examples, the inner layer 102 and/or the outer layer 108 can comprise a polymeric material having a modulus of elasticity of 400 Mpa or greater. Exemplary materials can include ultra-high-molecular-weight polyethylene (UHMWPE) (for example, Dyneema®), high-molecular-weight polyethylene (HMWPE), or polyether ether ketone (PEEK). With regard to the inner layer 102 in particular, such low coefficient of friction materials can facilitate passage of the prosthetic device through the lumen 112. Other suitable materials for the inner and outer layers can include polyimide, polytetrafluoroethylene (PTFE), expanded polytetrafluoroethylene (PTFE), ethylene tetrafluoroethylene (ETFE), nylon, polyethylene, polyamide, polyether block amide (for example, Pebax), and/or combinations of any of the herein. In some examples, the sheath 8 can include a lubricious liner on the inner surface of the inner layer 102. Examples of suitable lubricious liners include materials that can further reduce the coefficient of friction of the inner layer 102, such as PTFE, polyethylene, polyvinylidene fluoride, and combinations thereof. Suitable materials for a lubricious liner also include other materials desirably having a coefficient of friction of 0.1 or less.

Additionally, some examples of the sheath 8 can include an exterior hydrophilic coating on the outer surface of the outer layer 108. Such a hydrophilic coating can facilitate insertion of the sheath 8 into a patient's vessel, reducing potential damage. Examples of suitable hydrophilic coatings include the Harmony™ Advanced Lubricity Coatings and other Advanced Hydrophilic Coatings available from SurModics, Inc., Eden Prairie, MN. DSM medical coatings (available from Koninklijke DSM N.V, Heerlen, the Netherlands), as well as other hydrophilic coatings (for example, PTFE, polyethylene, polyvinylidene fluoride), are also suitable for use with the sheath 8. Such hydrophilic coatings may also be included on the inner surface of the inner layer 102 to reduce friction between the sheath and the delivery system, thereby facilitating use and improving safety. In some examples, a hydrophobic coating, such as Perylene, may be used on the outer surface of the outer layer 108 or the inner surface of the inner layer 102 in order to reduce friction.

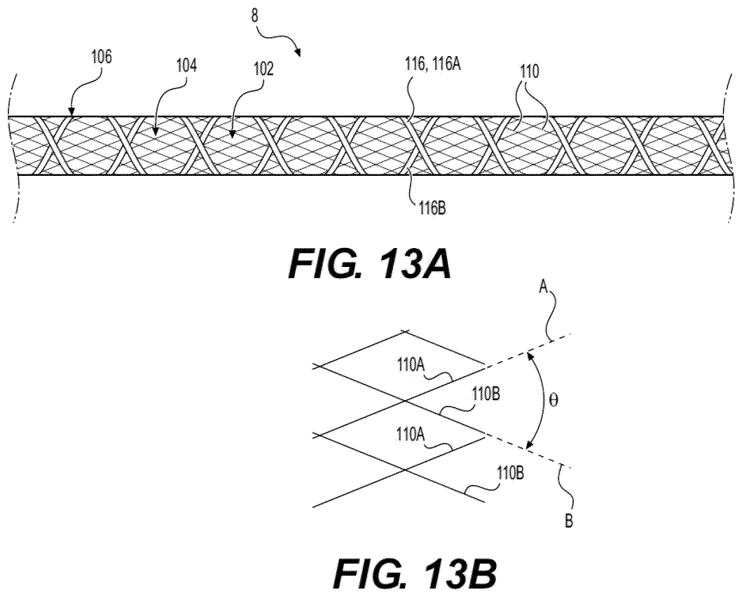

In certain examples, the second layer 104 can be a braided layer. FIGS. 13A and 13B illustrate the sheath 8 with the outer layer 108 removed to expose the elastic third layer 106. With reference to FIGS. 13A and 13B, the braided second layer 104 can comprise a plurality of members or filaments 110 (for example, metallic or synthetic wires or fibers) braided together. The braided second layer 104 can have any desired number of filaments 110, which can be oriented and braided together along any suitable number of axes. For example, with reference to FIG. 13B, the filaments 110 can include a first set of filaments 110A oriented parallel to a first axis A, and a second set of filaments 110B oriented parallel to a second axis B. The filaments 110A and 110B can be braided together in a biaxial braid such that filaments 110A oriented along axis A form an angle θ with the filaments 110B oriented along axis B. In certain examples, the angle θ can be from 5° to 70°, 10° to 60°, 10° to 50°, or 10° to 45°. In the illustrated example, the angle θ is 45°. In some examples, the filaments 110 can also be oriented along three axes and braided in a triaxial braid, or oriented along any number of axes and braided in any suitable braid pattern. The braided second layer 104 can extend along substantially the entire length L of the sheath 8, or alternatively, can extend only along a portion of the length of the sheath. In particular examples, the filaments 110 can be wires made from metal (for example, Nitinol, stainless steel, etc.), or any of various polymers or polymer composite materials, such as carbon fiber. In certain examples, the filaments 110 can be round, and can have a diameter of from 0.01 mm to 0.5 mm, 0.03 mm to 0.4 mm, or 0.05 mm to 0.25 mm. In some examples, the filaments 110 can have a flat cross-section with dimensions of 0.01 mm×0.01 mm to 0.5 mm×0.5 mm, or 0.05 mm×0.05 mm to 0.25 mm×0.25 mm. In one example, filaments 110 having a flat cross-section can have dimensions of 0.1 mm×0.2 mm. However, other geometries and sizes are also suitable for certain examples. If braided wire is used, the braid density can be varied. Some examples have a braid density of from ten picks per inch to eighty picks per inch, and can include eight wires, sixteen wires, or up to fifty-two wires in various braid patterns. In some examples, the second layer 104 can be laser cut from a tube, or laser-cut, stamped, punched, etc., from sheet stock and rolled into a tubular configuration. The second layer 104 can also be woven or knitted, as desired.

The third layer 106 can be a resilient, elastic layer (also referred to as an elastic material layer). In certain examples, the elastic third layer 106 can be configured to apply radially inward force to the underlying inner layer 102 and second layer 104 in a radial direction (for example, toward the central axis 114 of the sheath) when the sheath expands beyond its natural diameter by passage of the delivery apparatus through the sheath. Stated differently, the elastic third layer 106 can be configured to apply encircling/radially inward pressure to the layers of the sheath beneath the elastic third layer 106 to counteract expansion of the sheath. The radially inwardly directed force is sufficient to cause the sheath to collapse radially back to its unexpanded state after the delivery apparatus is passed through the sheath.

In the illustrated example, the elastic third layer 106 can comprise one or more members configured as strands, ribbons, or bands 116 helically wrapped around the braided second layer 104. For example, in the illustrated example the elastic third layer 106 comprises two elastic bands 116A and 116B wrapped around the braided second layer 104 with opposite helicity, although the elastic layer may comprise any number of bands depending upon the desired characteristics. The clastic bands 116A and 116B can be made from, for example, any of a variety of natural or synthetic elastomers, including silicone rubber, natural rubber, any of various thermoplastic elastomers, polyurethanes such as polyurethane siloxane copolymers, urethane, plasticized polyvinyl chloride (PVC), styrenic block copolymers, polyolefin elastomers, etc. In some examples, the elastic layer can comprise an elastomeric material having a modulus of elasticity of 200 Mpa or less. In some examples, the elastic third layer 106 can comprise a material exhibiting an elongation to break of 200% or greater, or an elongation to break of 400% or greater. The elastic third layer 106 can also take other forms, such as a tubular layer comprising an elastomeric material, a mesh, a shrinkable polymer layer such as a heat-shrink tubing layer, etc. In lieu of, or in addition to, the clastic third layer 106, the sheath 8 may also include an elastomeric or heat-shrink tubing layer around the outer layer 108. Examples of such elastomeric layers are disclosed in U.S. Publication No. 2014/0379067, U.S. Publication No. 2016/0296730, and U.S. Publication No. 2018/0008407, which are incorporated herein by reference. In some examples, the elastic third layer 106 can also be radially outward of the polymeric outer layer 108.

In certain examples, one or both of the inner layer 102 and/or the outer layer 108 can be configured to resist axial elongation of the sheath 8 when the sheath expands. More particularly, one or both of the inner layer 102 and/or the outer layer 108 can resist stretching against longitudinal forces caused by friction between a prosthetic device and the inner surface of the sheath 8 such that the length L remains substantially constant as the sheath expands and contracts. As used herein with reference to the length L of the sheath, the term “substantially constant” means that the length L of the sheath increases by not more than 1%, by not more than 5%, by not more than 10%, by not more than 15%, or by not more than 20%. Meanwhile, with reference to FIG. 13B, the filaments 110A and 110B of the braided second layer 104 can be allowed to move angularly relative to each other such that the angle θ changes as the sheath expands and contracts. This, in combination with the longitudinal ridges 126 (folds) in the inner layer 102 and outer layer 108, can allow the lumen 112 of the sheath to expand as a prosthetic device is advanced through it.

For example, the inner layer 102 and the outer layer 108 can be heat-bonded during the manufacturing process such that the braided second layer 104 and the elastic third layer 106 are encapsulated between the inner layer 102 and outer layer 108. More specifically, in certain examples the inner layer 102 and the outer layer 108 can be adhered to each other through the spaces between the filaments 110 of the braided second layer 104 and/or the spaces between the elastic bands 116. The inner layer 102 and outer layer 108 can also be bonded or adhered together at the proximal and/or distal ends of the sheath. In certain examples, the inner layer 102 and outer layer 108 are not adhered to the filaments 110. This can allow the filaments 110 to move angularly relative to each other, and relative to the inner layer 102 and outer layer 108, allowing the diameter of the braided second layer 104, and thereby the diameter of the sheath, to increase or decrease. As the angle θ between the filaments 110A and 110B changes, the length of the braided second layer 104 can also change. For example, as the angle θ increases, the braided second layer 104 can foreshorten, and as the angle θ decreases, the braided second layer 104 can lengthen to the extent permitted by the areas where the inner layer 102 and outer layer 108 are bonded. However, because the braided second layer 104 is not adhered to the inner layer 102 and outer layer 108, the change in length of the braided layer that accompanies a change in the angle θ between the filaments 110A and 110B does not result in a significant change in the length L of the sheath.

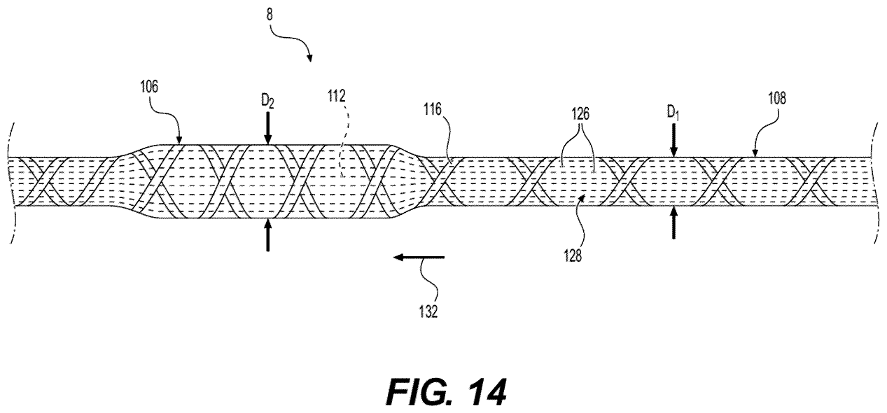

FIG. 14 illustrates radial expansion of the sheath 8 as a prosthetic device (for example, implant 12) is passed through the sheath 8 in the direction of arrow 132 (for example, distally). As the prosthetic device (implant 12) is advanced through the sheath 8, the sheath can resiliently expand to a second diameter D2 that corresponds to a size or diameter of the prosthetic device. As the prosthetic device (implant 12) is advanced through the sheath 8, the prosthetic device can apply longitudinal force to the sheath in the direction of motion by virtue of the frictional contact between the prosthetic device and the inner surface of the sheath. However, as noted herein, the inner layer 102 and/or the outer layer 108 can resist axial elongation such that the length L of the sheath remains constant, or substantially constant. This can reduce or prevent the braided layer second 104 from lengthening, and thereby constricting the lumen 112.

Meanwhile, the angle θ between the filaments 110A and 110B can increase as the sheath expands to the second diameter D2 to accommodate the prosthetic valve. This can cause the braided second layer 104 to foreshorten. However, because the filaments 110 are not engaged or adhered to the inner layer 102 and outer layer 108, the shortening of the braided second layer 104 attendant to an increase in the angle θ does not affect the overall length L of the sheath. Moreover, because of the longitudinally-extending ridges 126 (folds) formed in the inner layer 102 and outer layer 108, the inner layer 102 and outer layer 108 can expand to the second diameter D2 without rupturing, in spite of being relatively thin and relatively non-clastic. In this manner, the sheath 8 can resiliently expand from its natural diameter D1 to a second diameter D2 that is larger than the diameter D1 as a prosthetic device is advanced through the sheath, without lengthening, and without constricting. Thus, the force required to push the prosthetic implant through the sheath is significantly reduced.

Additionally, because of the radial force applied by the elastic third layer 106, the radial expansion of the sheath 8 can be localized to the specific portion of the sheath occupied by the prosthetic device. For example, with reference to FIG. 14, as the prosthetic device (implant 12) moves distally through the sheath 8, the portion of the sheath immediately proximal to the prosthetic device (for example, implant 12) can radially collapse back to the initial diameter D1 under the influence of the clastic third layer 106. The inner layer 102 and outer layer 108 can also buckle as the circumference of the sheath is reduced, causing the folds/ridges 126 and the valleys 128 to reform. This can reduce the size of the sheath required to introduce a prosthetic device of a given size. Additionally, the temporary, localized nature of the expansion can reduce trauma to the blood vessel into which the sheath is inserted, along with the surrounding tissue, because only the portion of the sheath occupied by the prosthetic device expands beyond the sheath's natural diameter and the sheath collapses back to the initial diameter once the device has passed. This limits the amount of tissue that must be stretched in order to introduce the prosthetic device, and the amount of time for which a given portion of the vessel must be dilated.

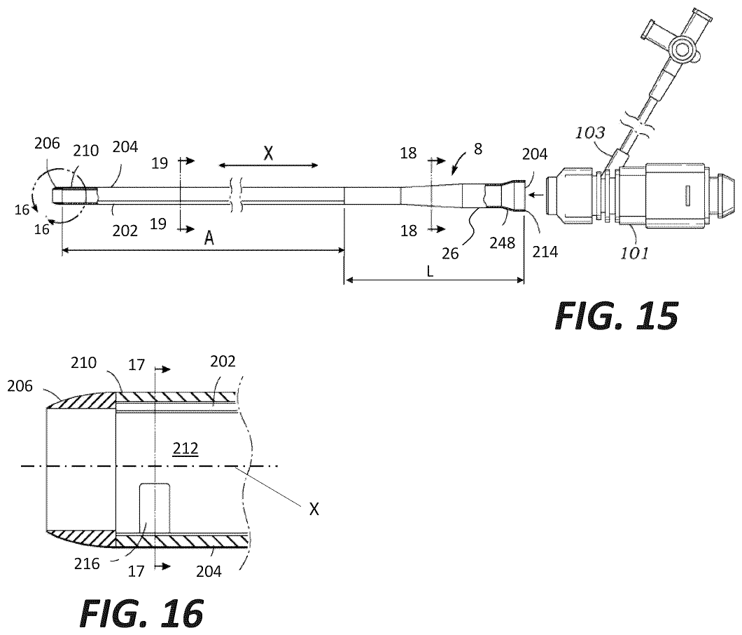

In another example layered sheath 8 structure, FIGS. 15-23 illustrate various features of the coaxial layered structure of the expandable sheath 8 of FIG. 1 according to another example. Similar reference numbers are used to describe like elements. It is to be understood that the variations (for example, materials and alternate configurations) described herein with reference to FIGS. 11-14 can also apply to the example shown in FIGS. 15-23. Furthermore, the variations described herein with reference to FIGS. 15-23 can also be applied to the sheath described in FIGS. 11-14.

Similar to various aspects of the sheath 8 described herein in reference to FIGS. 11-14, the sheath 8 of FIGS. 15-23 includes a plurality of layers. For example, the sheath 8 illustrated in FIGS. 15-23, also includes an inner layer 202 and an outer layer 204 disposed around the inner layer 202. The inner layer 202 can define a lumen 212 through which the delivery apparatus travels into the patient's vessel in order to deliver, remove, repair, and/or replace a prosthetic device, moving in a direction along the longitudinal axis X. Similar to the sheath illustrated in FIGS. 11-14, as the prosthetic device passes through the sheath 8, the sheath 8 locally expands from a first, resting/unexpanded diameter to a second, expanded diameter to accommodate the prosthetic device. After the prosthetic device passes through a particular location of the sheath 8, each successive expanded portion or segment of the sheath 8 at least partially returns to the smaller, resting/unexpanded diameter. In this manner, the sheath 8 can be considered self-expanding, in that it does not require use of a balloon, dilator, and/or obturator to expand.

Similar to the examples herein, the inner layer 102 and the outer layer 204 can comprise any suitable materials. Suitable materials for the inner layer 202 include polytetrafluoroethylene (PTFE), ethylene tetrafluoroethylene (ETFE), nylon, polyethylene, polyether block amide (for example, Pebax), and/or combinations thereof. In a particular example, the inner layer 202 can comprise a lubricious, low-friction, or hydrophilic material, such as PTFE. Such low coefficient of friction materials can facilitate passage of the prosthetic device through the lumen defined by the inner layer 202. In some examples, the inner layer 202 can have a coefficient of friction of less than about 0.1. Some examples of the sheath 8 can include a lubricious liner on the inner surface of the inner layer 202. Examples of suitable lubricious liners include materials that can further reduce the coefficient of friction of the inner layer 202, such as PTFE, polyethylene, polyvinylidene fluoride, and combinations thereof. Suitable materials for a lubricious liner also include other materials desirably having a coefficient of friction of about 0.1 or less.

Suitable materials for the outer layer 204 include nylon, polyethylene, Pebax, HDPE, polyurethanes (for example, Tecoflex), and other medical grade materials. In an example, the outer layer 204 can comprise high density polyethylene (HDPE) and Tecoflex (or other polyurethane material) extruded as a composite. In some examples, the Tecoflex can act as an adhesive between the inner layer 202 and the outer layer 204 and may only be present along a portion of the inner surface of the outer layer 204. Other suitable materials for the inner and outer layers are also disclosed in U.S. Pat. Nos. 8,690,936 and 8,790,387, which are incorporated herein by reference.

Additionally, some examples of the sheath 8 include an exterior hydrophilic coating on the outer surface of the outer layer 204. Such a hydrophilic coating can facilitate insertion of the sheath 100 into a patient's vessel. Examples of suitable hydrophilic coatings include the Harmony™ Advanced Lubricity Coatings and other Advanced Hydrophilic Coatings available from SurModics, Inc., Eden Prairie, MN. DSM medical coatings (available from Koninklijke DSM N.V, Heerlen, the Netherlands), as well as other hydrophilic coatings (for example, PTFE, polyethylene, polyvinylidene fluoride), are also suitable for use with the sheath 100.

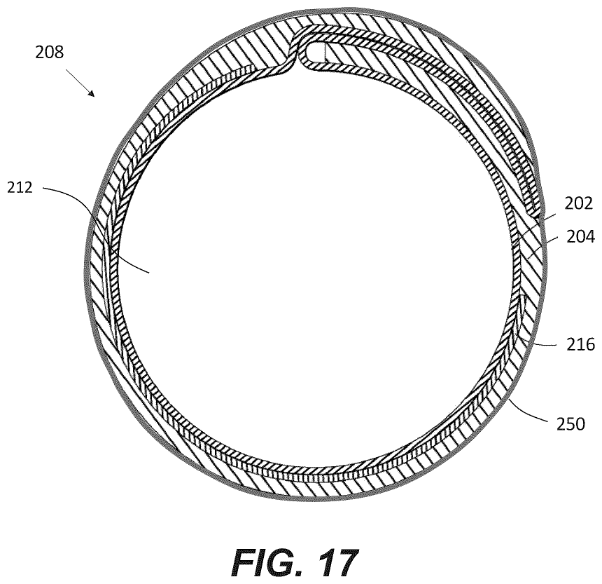

FIG. 16 provides a partial cross-section of the distal end of the sheath 8 along section line 16-16 identified in FIG. 15. As described herein, the sheath 8 can be inserted into a vessel (for example, the femoral or iliac arteries) by passing through the skin of patient, such that a soft tip portion 206 at the distal end 210 of the sheath 8 is inserted into the vessel. As best seen in FIG. 16, the soft tip portion 206 can comprise, in some examples, low density polyethylene (LDPE) and can be configured to minimize trauma or damage to the patient's vessels as the sheath is navigated through the vasculature. For example, the soft tip portion 206 can be slightly tapered to facilitate passage through the vessels. The soft tip portion 206 can be secured to the distal end 210 of the sheath 8, such as by thermally bonding the soft tip portion 206 to the inner and outer layers of the sheath 8. Such a soft tip portion 206 can be provided with a lower hardness than the other portions of the sheath 8. In some examples, the soft tip portion 206 can have a Shore hardness from about 25 D to about 40 D. The soft tip portion 206 is configured to be radially expandable to allow a prosthetic device to pass through the distal opening of the sheath 208. For example, the soft tip portion 206 can be formed with a weakened portion, such as an axially extending score line or perforated line that is configured to split and allow the soft tip portion 206 to expand radially when the prosthetic device passes therethrough.

FIG. 17 shows a cross-section view of the sheath 8 taken near the distal end 210 of the sheath 8 as indicated by section line 17-17 in FIG. 16. As illustrated in FIGS. 16 and 17, the sheath 8 can include at least one radiopaque filler or marker, such as a discontinuous, or C-shaped, band/marker 216 positioned near the distal end 210 of the sheath 8. The marker 216 can be associated with the inner layer 202 and/or outer layer 204 of the sheath 8. For example, as shown in FIG. 17, the marker 216 can be positioned between the inner layer 202 and the outer layer 204. In alternative examples, the marker 216 can be associated with the outer surface of the outer layer 204. In some examples, the marker 216 can be embedded or blended within the inner layer 202 or outer layer 204.

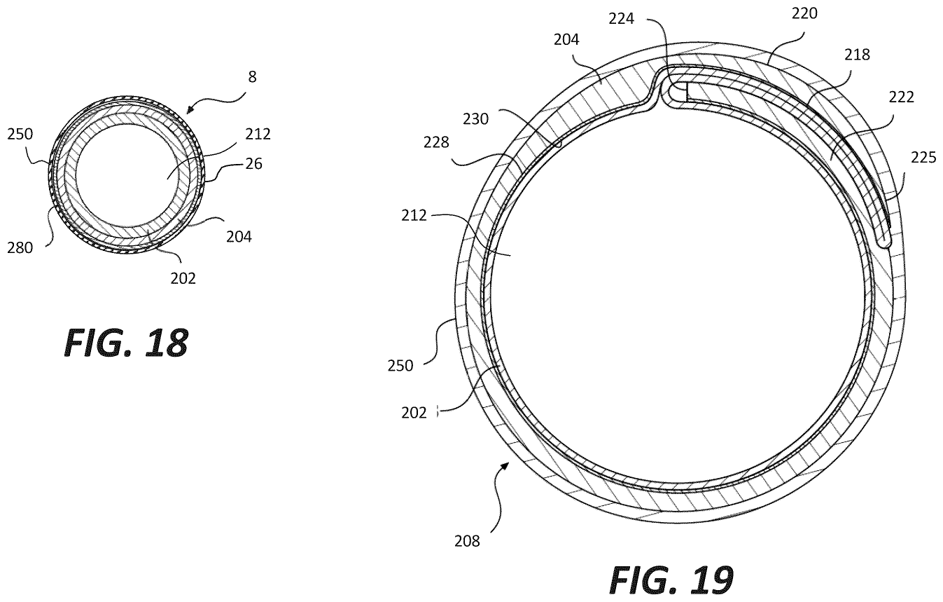

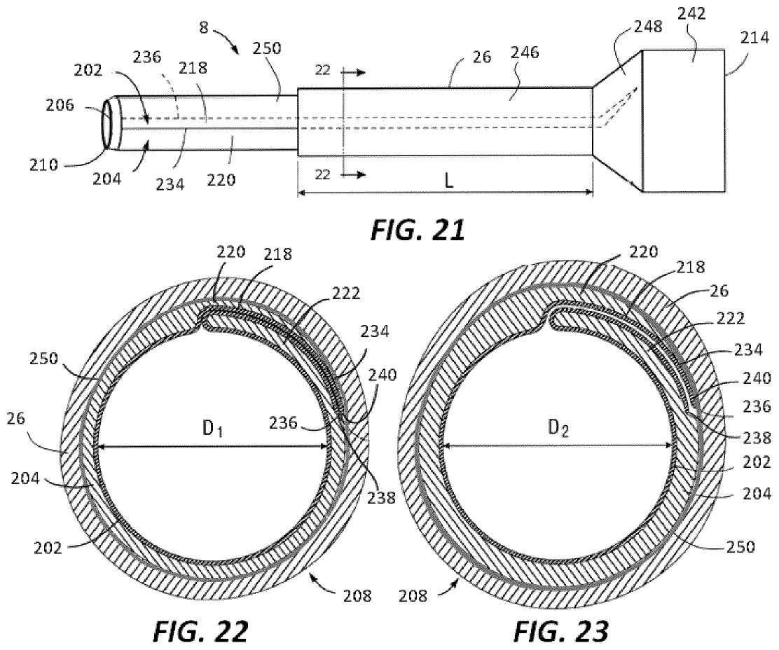

FIGS. 18 and 19 show additional cross-sections taken at different points along the sheath 208. FIG. 18 shows a cross-section of a segment of the sheath near the proximal end 214 of the sheath 8, as indicated by section line 18-18 in FIG. 15. At this location, the sheath 8 includes the inner layer 202, outer layer 204, elastic outer layer 250/outer jacket, and the strain relief layer 26. At this location, near the proximal end of the sheath 8, the inner layer 202 and outer layer 204 are substantially tubular. Here the inner layer 202 and outer layer 204 can be formed without any slits or folded portions in the layers. By contrast, as described herein, the inner layer 202 and outer layer 204 at different locations along the sheath 8 (for example, at the point indicated by section line 19-19 in FIG. 15 and/or the point indicated by section line 22-22 in FIG. 21) can have a different configuration.

As shown in FIG. 19, the inner layer 202 can be arranged to form a substantially cylindrical lumen 212 therethrough. Inner layer 202 can include one or more folded portions 218. In the example shown in FIG. 19, inner layer 202 is arranged to have one folded portion 218 that can be positioned on either side of the inner layer 202. Inner layer 202 can be continuous, in that there are no breaks, slits, or perforations in inner layer 202. Outer layer 204 can be arranged in an overlapping fashion such that an overlapping portion 220 overlaps at least a part of the folded portion 218 of the inner layer 202. As shown in FIG. 19, the overlapping portion 220 also overlaps an underlying portion 222 of the outer layer 204. The underlying portion 222 can be positioned to underlie both the overlapping portion 220 of the outer layer 204, as well as the folded portion 218 of the inner layer 202. Thus, the outer layer 204 can be discontinuous, in that it includes a slit or a cut in order to form the overlapping portion 220 and the underlying portion 222. In other words, a first edge 224 of the outer layer 204 is spaced apart from a second edge 225 of the outer layer 204 so as not to form a continuous layer.

As shown in FIG. 19, the sheath 8 can also include a thin layer of bonding or adhesive material 228 positioned between the inner layer 102 and the outer layer 204. In an example, the adhesive material 228 can comprise a polyurethane material such as Tecoflex. The adhesive material 228 can be positioned on an inner surface of at least a portion of the outer layer 204 so as to provide adhesion between selected portions of the inner layer 202 and outer layer 204. For example, the outer layer 204 may only include a Tecoflex layer (adhesive material 228) around the portion of the inner surface 230 that faces the lumen-forming portion of the inner layer 202. In other words, the Tecoflex layer (adhesive material 228) can be positioned so that it does not contact the folded portion 218 of the inner layer 202. In some examples, the Tecoflex can be positioned in different configurations as desired for the particular application. For example, as shown in FIG. 19, the Tecoflex layer can be positioned along the entire inner surface 230 of the outer layer 204. In an alternative example, the Tecoflex layer can be applied to the outer surface of the inner layer 202 instead of the inner surface of the outer layer 204. The Tecoflex layer can be applied to all or selected portions on the inner layer 202; for example, the Tecoflex layer can be formed only on the portion of the inner layer 202 that faces the lumen-forming portion of the outer layer 204 and not on the folded portion 218. The configuration of FIG. 19 allows for radial expansion of the sheath 208 as an outwardly directed radial force is applied from within (for example, by passing a medical device such as a prosthetic heart valve through the lumen 212). As radial force is applied, the folded portion 218 can at least partially separate, straighten, and/or unfold, and/or the overlapping portion 220 and the underlying portion 222 of the outer layer 204 can slide circumferentially with respect to one another, thereby allowing the diameter of lumen 212 to enlarge.

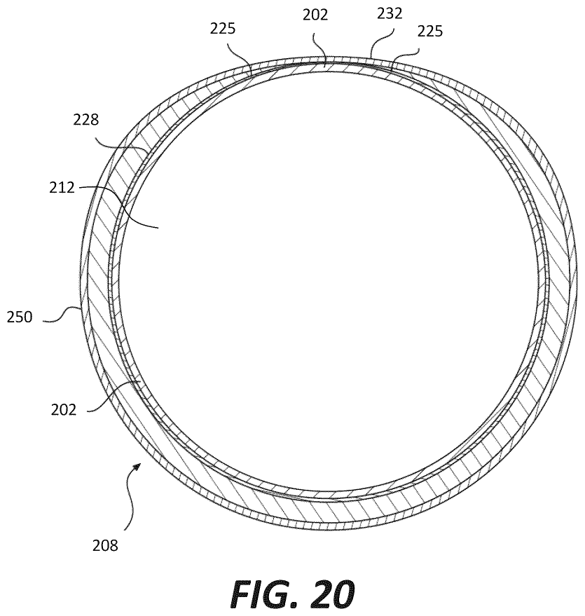

In this manner, the sheath 208 is configured to expand from a resting/unexpanded configuration (FIG. 19) to an expanded configuration shown in FIG. 20. In the expanded configuration, as shown in FIG. 20, an annular gap 232 can form between the longitudinal edges of the overlapping portion 220 and the underlying portion 222 of the outer layer 204. As the sheath 208 expands at a particular location, the overlapping portion 220 of the outer layer 204 can move circumferentially with respect to the underlying portion 222 as the folded portion 218 of the inner layer 202 unfolds. This movement can be facilitated by the use of a low-friction material for inner layer 202, such as PTFE. Further, the folded portion 218 can at least partially separate and/or unfold to accommodate a medical device having a diameter larger than that of lumen 212 in the resting/unexpanded configuration. As shown in FIG. 20, in some examples, the folded portion of the inner layer 202 can completely unfold, so that the inner layer 202 forms a cylindrical tube at the location of the expanded configuration.

Similar to the example sheath 8 in FIG. 14, the sheath 8 is configured to locally expands at a particular location corresponding to the location of the medical device along the length of the lumen 212, and then locally contracts once the medical device has passed that particular location. Thus, a bulge may be visible, traveling longitudinally along the length of the sheath 8 as a medical device is introduced through the sheath 8, representing continuous local expansion and contraction as the device travels the length of the sheath 8. Each segment of the sheath 8 will locally contract after removal of any radial outward force such that the sheath 8 at least partially returns to the original resting/unexpanded diameter of lumen 212. Similar to the example sheath described herein, an elastic outer layer 250 can (optionally) be provided along the sheath 8, urging the inner layer 202 and outer layer 204 back towards the unexpanded configuration.

The inner layer 202 and outer layer 204 of sheath 8 can be configured having the folded portion 218 as shown in FIG. 19 along at least a portion of the length of the sheath 208. In some examples, the inner layer 202 and outer layer 204 can be configured as shown in FIG. 19 along the length A (FIG. 15) such that the folded portion 218 extends from a location adjacent the soft tip portion 206 to a location closer to the proximal end 214 of the sheath 8, adjacent and/or under the distal end of the strain relief layer 26. In this matter, the sheath 8 is expandable and contractable only along a portion of the length of the sheath corresponding to length A (which typically corresponds to the section of the sheath inserted into the narrowest section of the patient's vasculature).

In some examples, the folded portion 218 portion extends from a location adjacent the soft tip portion 206 under the strain relief layer 26, as illustrated in FIG. 21. In this example, the folded structure of the inner layer 202 extends from the soft tip portion 206, under the strain relief layer 26 and along the tapered portion 248 of the strain relief layer 26.

FIGS. 22 and 23 illustrate cross-section views of the sheath 8 taken along the strain relief layer 26 at section line 22-22 in FIG. 21. In this example, the folded portion 218 of the inner layer 202 extends under the strain relief layer 26. FIG. 22 shows a cross-section of the sheath 8 in a resting/unexpanded configuration having an inner diameter D1. FIG. 23 shows a cross-section of the sheath 208 in a (partially) expanded configuration, having an inner diameter D2, where D2 is greater than D1.

As shown in FIGS. 22-23, in some examples, the overlapping portion 220 does not overlap the entire folded portion 218 of the inner layer 202, and thus a portion of the folded portion 218 can be directly adjacent to the strain relief layer 26 in locations where the strain relief layer 26 is present. In locations where the strain relief layer 26 is not present, part of the folded portion 218 may be visible from the outside of the sheath 8, as seen in FIG. 21 (and/or visible through an elastic outer layer 250 described in more detail herein). In these examples, the sheath 8 can include a longitudinal seam 234 where the overlapping portion 220 terminates at the folded portion 218. In use, the sheath 8 can be positioned such that the seam 234 is posterior to the point of the sheath that is 180 degrees from the seam 234 (for example, facing downward in the view of FIG. 21). As shown in FIG. 21, the seam 234 need not extend the entire length of the sheath 8, and end at a transition point between portions of the sheath having a folded inner layer and portions of the sheath not having a folded inner layer.

In some examples, the folded portion 218 can include a weakened portion 236, such as a longitudinal perforation, score line, and/or slit, along at least a portion of the length of the inner layer 202. The weakened portion 236/slit allows for the two adjacent ends 238, 240 of the folded portion 218/inner layer 202 to move relative to one another as the sheath 8 expands to the expanded configuration shown in FIG. 23. For example, the sheath 8 locally expands as a medical device is inserted therethrough, causing the weakened portion 236 to split/separate.

In each of the example sheaths 8 described herein, the sheath 8 may include an elastic outer layer 250 that expands with the sheath 8. The clastic outer layer 250 can provide an inwardly directed radial force that directs the sheath towards a folded/unexpanded configuration. Similar to the strain relief layer 26, elastic outer layer 250 can also provide hemostasis (for example, prevent blood loss during implantation of the prosthetic device).

The clastic outer layer 250 can be positioned around at least a portion of the strain relief layer 26, outer layer 108, 204 and/or the inner layers of the sheath 8. As illustrated in FIGS. 21-23, the outer layer 250 can surround the entire circumference of outer layer 204, and can extend longitudinally along any portion of the length of the sheath 8, including along (over or under) the strain relief layer 26. The elastic outer layer 250 extends for a length along at least a portion of the main body of the sheath 8. In some examples, the elastic outer layer 250 extends to a point adjacent the distal end 210, or can extend all the way to the distal end 210 of sheath 8. For example, the clastic outer layer 250 extends over the entire length of the sheath 8.

As shown in FIGS. 17-20, 22 and 23, the clastic outer layer 250 can be a continuous tubular layer, without slits or other discontinuities. The clastic outer layer 250 extends between strain relief layer 26 and the outer surface of the outer layer 204. In some examples, the clastic outer layer 250 extends over the outer surface of the strain relief layer 26 and the outer surface of the outer layer 204. In further examples, the elastic outer layer 250 extends both over the strain relief layer 26 and/or between the outer layer of the sheath 8 and the strain relief layer 26.

The clastic outer layer 250 can comprise any pliable, clastic material(s) that expand and contract, preferably with a high expansion ratio. Preferably, the materials used can include low durometer polymers with high elasticity, such as Pebax, polyurethane, silicone, and/or polyisoprene. Materials for the elastic outer layer 250 can be selected such that it does not impede expansion of the inner and outer layers of the sheath 8. The clastic outer layer 250 can have a thickness ranging from, for example, about 0.001″ to about 0.010.″ In some examples, the elastic outer layer 250 can have a thickness of from about 0.003″ to about 0.006.″ The elastic outer layer 250 can be configured to stretch and expand as the sheath expands, as shown in the expanded configuration in FIG. 20.

As illustrated in FIGS. 2, 15, 21 and 24, the sheath 8 in each of the examples described herein may include a strain relief layer 26. The strain relief layer 26 is provided adjacent the proximal end of the sheath 8 and extends along/over the outer surface of the sheath 8. In some examples, the strain relief layer 26 is provided over the outer layer 108, 204 of the sheath 8. The strain relief layer 26 forms a smooth transition between the sheath hub 20 and the sheath 8 and facilitates mating of the sheath 8 with the sheath hub 20.

Additionally, and as will be described in more detail herein, the strain relief layer 26 provides a region of higher durometer or stiffness that restricts expansion of the underlying sheath layers. This helps to ensure hemostasis between the portions of the sheath 8 inside the patient and the sheath hub (external to the patient). The increased durometer and/or stiffness along the strain relief layer 26 prevents blood from flowing between the various layers of the sheath 8 exterior to the patient during the procedure, helping to withstand the blood pressure that would otherwise cause the sheath to “balloon up” with body fluid/blood. Additionally, the strain relief layer 26 can be sized and configured to form a seal with the patient's artery when inserted, such that blood is substantially prevented from flowing between the strain relief layer 26 and the vessel wall. For example, although the strain relief layer 26 does not extend all the way to the distal end 210 of the sheath 8, the strain relief layer 26 can extend distally enough along the sheath 8 that when the sheath 8 is fully inserted into the patient, a portion of the strain relief layer 26 extends through and seals against the arteriotomy site.

As described herein, the strain relief layer 26 is provided over the outer layer 108, 204 of the sheath 8. The strain relief layer 26 can be bonded to the outer layer 108, 204 to prevent the strain relief layer 26 from sliding over the outer layer and “bunching up” in response to the friction forces applied by the surrounding tissue during insertion of the sheath 8 into the patient's vasculature. For example, the strain relief layer 26 can be bonded at the proximal end and/or distal end of the outer layer 108, 204. At the proximal and distal ends, the strain relief layer 26 can be bonded to the outer layer 204 around the full circumference of the outer layer. At the distal end of the sheath 208, the strain relief layer 26 can alternatively be bonded to the inner layer(s) of the sheath 8. For example, the strain relief layer 26 can be bonded to the distal end surface of the inner layer 102, 202.

FIGS. 18, 22 and 23 illustrate cross-section views of the sheath 8 along the strain relief layer 26. FIG. 18 shows a cross-section of a segment of the sheath near the proximal end 214 of the sheath 8, as indicated by section line 18-18 in FIG. 15. Similarly, FIGS. 22 and 23 show cross-section segments of various example sheaths near the proximal end 214 of the sheath 8 and closer to the distal end of the strain relief layer 26, as indicated by section line 22-22 in FIG. 21. As illustrated in each of FIGS. 15-23, the sheath 8 at this location can comprise an inner layer (liner) 202, outer layer 204, adhesive material 228, an optional elastic outer layer 250, and the strain relief layer 26.

The strain relief layer 26 extends circumferentially around at least a portion of the inner layer 202 and outer layer 204. The strain relief layer 26 extends from the proximal end 214 of the sheath 8 towards the distal end 210 of the sheath 8. As shown in FIG. 21 (and FIG. 15), the strain relief layer 26 extends for a length L along at least a portion of the main body of the sheath 8. In some examples, the strain relief layer 26 extends to a point adjacent the distal end 210, or can extend all the way to the distal end 210 of sheath 8. In some examples, the longitudinal length L of the strain relief layer 26 can range from about 10 cm to the entire length of the sheath 8.

The strain relief layer 26 extends to/adjacent the proximal end 214 of the sheath 8 and provides a compression fit over the distal end of the sheath hub 20 thereby coupling the sheath 8 to the sheath hub 20. Additionally, or alternatively, the strain relief layer 26 secured between the sheath hub 20 and the sheath hub cap 22 or other fastening device for by coupling the proximal end of the sheath to the sheath hub 20. In some examples, the strain relief layer 26 does not extend all the way to the proximal end 214 of the sheath 208.

It is understood that strain relief layer 26, as shown herein, can have similar composition and characteristics of the inner and outer layers as disclosed herein. Various compositions are disclosed, for example, in Application No. PCT/US2021/301275, entitled “Expandable sheath for introducing an endovascular delivery device into a body,” the disclosure of which is herein incorporated by reference.

The strain relief layer 26 can comprise any lubricious, low-friction, and/or relatively non-elastic material. Preferably the materials used can include high durometer polymers, with low elasticity. In some examples, the strain relief layer 26 is composed of the same and/or similar material to the inner layer 202 and/or outer layer 204. For example, as described herein regarding the inner layer 102 and/or outer layer 108, exemplary materials can include polyurethane (for example, high density polyethylene), ultra-high-molecular-weight polyethylene (UHMWPE) (for example, Dyneema®), high-molecular-weight polyethylene (HMWPE), or polyether ether ketone (PEEK). Other suitable materials strain relief layer 26 can include polyimide, polytetrafluoroethylene (PTFE), expanded polytetrafluoroethylene (ePTFE), ethylene tetrafluoroethylene (ETFE), nylon, polyethylene, polyamide, polyether block amide (for example, Pebax), and/or combinations of any of the herein. Materials for the strain relief layer 26 can be selected such that it impedes expansion of the underlying layers of the sheath 8.

The strain relief layer 26 can have a thickness ranging from, for example, about 0.001″ to about 0.010.″ In some examples, the strain relief layer 26 can have a thickness of from about 0.003″ to about 0.006.″ The wall thickness is measured radially between the inner surface of the strain relief layer 26 and the outer surface of the strain relief layer 26.

In alternative examples, the material composition and/or wall thickness can change along the length of the strain relief layer 26. For example, the strain relief layer 26 can be provided with one or more segments, where the composition and/or thickness changes from segment to segment. For example, the Durometer rating of the composition can change along the length of the strain relief layer 26 such that segments near the proximal end comprise a stiffer material or combination of materials, while segments near the distal end comprise a softer material or combination of materials. Similarly, the wall thickness of the strain relief layer 26 in segments near the proximal end can be thicker/greater than the wall thickness of the clastic outer layer 250 near the distal end.

As illustrated in FIGS. 15, 21 and 24, the strain relief layer 26 has a proximal end and a distal end and a central lumen extending longitudinally therethrough. The strain relief layer 26 includes a generally tubular-shaped proximal portion 242 adjacent the proximal end of the strain relief layer 26, and a generally tubular-shaped distal portion 246 adjacent the distal end of the strain relief layer 26. The strain relief layer 26 includes a frustoconical-shaped tapered portion 248 extending between the proximal portion 242 and the distal portion 246 of the strain relief layer 26, such that the diameter of the strain relief layer 26 at the proximal portion 242 is greater than the diameter of the strain relief layer 26 at the distal portion 246 of the strain relief layer 26. The tapered portion 248 and the flared proximal portion 242 help case the transition of the medical device/delivery system when passing between the larger diameter sheath hub 20 to the smaller diameter of the sheath 8.

As described herein, the strain relief layer 26 is made of a material that is stiffer than the other sheath 8 layers such that the strain relief layer 26 inhibits expansion of the portion of the sheath disposed along/under the strain relief layer 26. Because radial expansion is limited along the strain relief layer 26, higher push forces are necessary to advance the medical device through the central lumen of the sheath 8. In some examples, the highest push force through the sheath 8 are experienced near the proximal and distal ends of the sheath 8, through the strain relief layer 26 (for example, through the tapered portion of the strain relief layer), and at the proximal and distal ends of the strain relief layer 26. In addition, recent trends in heart valves including thicker PVL skirts has increased the crimped profile of the heart valve/delivery device and can lead to even higher push forces through the sheath 8, and particularly through the strain relief layer 26 and the distal opening of the sheath 8.

As described herein, it is desirable to reduce push forces required to insert the medical device/delivery system through the central lumen of the sheath 8. In some examples, the thickness and/or composition of the strain relief layer 26 and/or sheath 8 can be adjusted to improve the performance of the sheath 8 and to reduce the push force. In some examples, dilating or expanding the sheath 8 (or a portion thereof) before the medical device/delivery system is introduced helps to reduce the initial push force through the sheath. Pre-dilating the sheath 8 releases and/or loosens any bonding or adhesion of the sheath 8 layers that occurs during the manufacturing process, for example, bonding between the inner layer 202 and the outer layer 204, bonding between the folded portion 218 and outer layer 204, bonding between the inner/outer layers and the strain relief layer 26. Pre-dilating can also break or separate the weakened portion 236 of folded portion 218 of the inner layer 202, separating adjacent ends 238, 240 of the folded portion 218, as described herein and illustrated in FIG. 23. With the sheath 8 layers able to move freely with respect to the other, the medical device/delivery system is pushed through the sheath 8 lumen at a much lower force.

In some instances, the sheath 8 is pre-dilated by passing a relatively large dilator (for example, 22 French dilator) into the sheath 8 and through the strain relief layer 26. This can be done during sheath 8 preparation, prior to sheath 8 insertion into the patient and/or with the sheath 8 at least partially inserted into the patient. However, this method requires significant physical strength of the user (i.e., grip and arm strength) to advance the dilator through the sheath, and particularly through the strain relief layer 26. Additionally, it is challenging to control the dilation distance. In some instances, it can be important that the sheath 8 not be dilated beyond the distal end of the strain relief layer 26. Expanding/dilating the sheath 8 beyond the end of the strain relief layer 26 can cause irregular sheath 8 expansion and difficulty or vessel injury during insertion, movement and/or withdraw of the sheath 8 in the vasculature. Current methods for controlling the desired dilation length of the sheath 8 and/or strain relief layer 26 is prone to user error and/or inaccuracies because it relies on a user's visual observation of the dilator as it passes through the strain relief layer 26 and stopping advancement just when the portion of the sheath 8 beyond the distal end of the strain relief layer 26 starts to expand. This manual method is inherently difficult to train, difficult to enforce proper technique, and prone to errors.

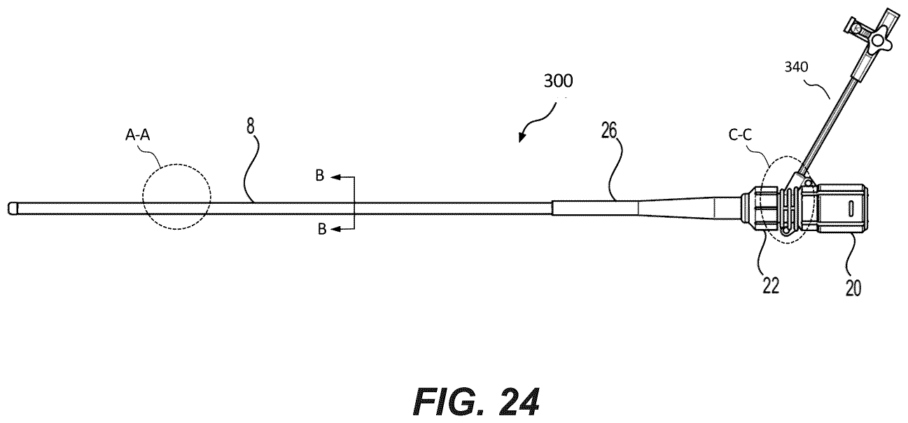

In the example sheath system described with respect to FIGS. 24-30, the structure of the sheath 8 can be modified compared to the sheath structures/layers described herein, to provide a self-expanding sheath allowing for controlled expansion along the length of the sheath 8. The devices, systems, and methods described herein provide an inflatable introducer sheath 8. The sheath 8 can be used individually, in lieu of the layered sheath structures described herein with respect to FIGS. 11-23. It is also contemplated, that the inflatable sheath 8 described herein can be provided within the central lumen of the sheath of FIGS. 11-23 to dilate/pre-dilate the sheath in advance of medical device delivery.

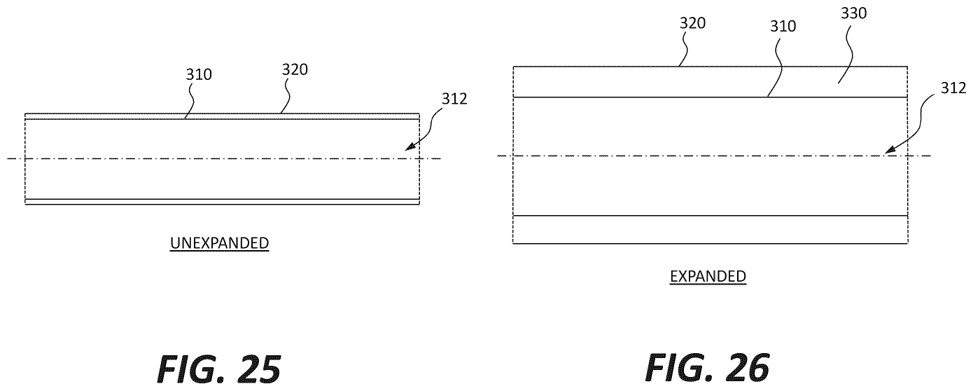

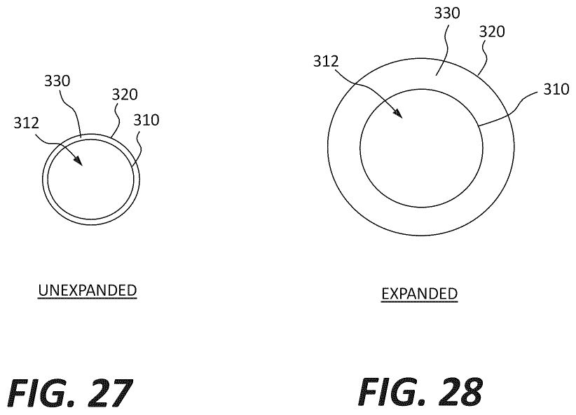

FIG. 24 shows an example inflatable sheath system 300. The inflatable sheath system 300 includes similar sheath hub 20 and connecting structure as described herein. The sheath system 300 can also include the include elastic outer layer 250/outer jacket and strain relief layer 26 as described herein. The differences between the internal structure of the sheath 8 (compared to the layered structure described herein in reference to FIGS. 11-23) is provided in more detail herein. FIGS. 25 and 26 provide partial (longitudinal) cross-sectional views of the sheath 8 of FIG. 24 taken along section line A-A. FIG. 25 shows the sheath 8 in an unexpanded configuration, and FIG. 26 shows the sheath 8 in an expanded configuration. FIGS. 27 and 28 provide partial (axial) cross-sectional views of the sheath 8 of FIG. 24 taken along section line B-B. FIG. 27 shows the sheath 8 in an unexpanded configuration, and FIG. 28 shows the sheath 8 in an expanded configuration.

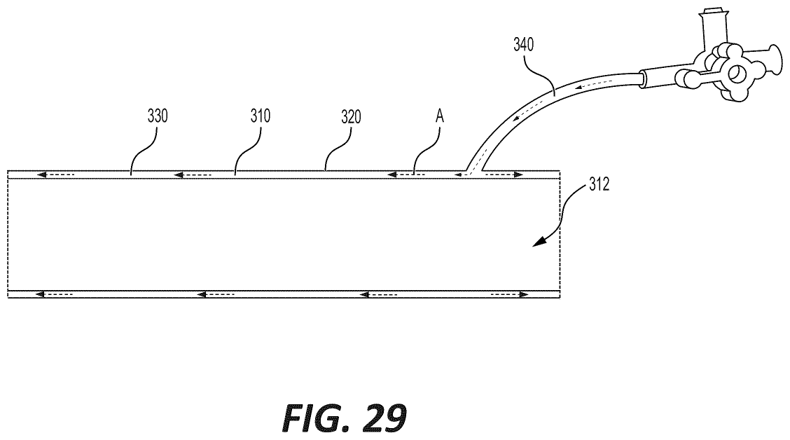

As shown in FIGS. 25-28, the sheath 8 includes an inner layer 310 defining the central lumen 312 of the sheath 8 extending therethrough. An outer layer 320 is provided over the inner layer 310 such that a fluid chamber 330 (for example, a fluid-bearing chamber) is defined between the inner layer 310 and outer layer 320. The sheath 8 includes an inflation port 340 in fluid communication with the fluid chamber 330. FIG. 29 provides a partial (longitudinal) cross-sectional view of the sheath 8 of FIG. 24 taken along section line C-C, with the sheath hub 20 structure omitted.

Upon inflation of the fluid chamber 330, at least a portion of the sheath 8 (for example, the inner layer 310 and/or the outer layer 320) expands from an unexpanded configuration (FIGS. 25, 27) in which the central lumen 312 has a first diameter to an expanded configuration (FIGS. 26, 28) in which the central lumen 312 has a second, larger, diameter. Similarly, the outer diameter of the outer layer 320 expands, thereby expanding the adjacent portion of the patient's vasculature. As will be described in more detail herein, the larger diameter central lumen 312 allows the push force required to advance a medical device through the sheath 8 to be reduced. In some examples, the larger outer diameter of the outer layer 320 can help to reduce curvature of patient vasculature.

Once the medical device is delivered, the sheath 8 is deflated for removal. Upon deflation of the fluid chamber 330 (for example, upon removal of the inflation fluid), at least a portion of the sheath 8 (for example, the inner layer 310 and/or the outer layer 320) returns toward the unexpanded configuration. For example, the diameter of the central lumen 312 and/or the outer diameter of the outer layer 320 return toward their corresponding unexpanded diameters.

In the expanded configuration, the inner layer 310 and outer layer 320 both expand from a first diameter to a second, larger, diameter. In some examples, the expanded outer diameter of the outer layer 320 ranges from 14 French to 24 French. Similarly, the expanded inner diameter of the central lumen 312 ranges from 14 French to 24 French. For example, the expanded inner diameter of the central lumen 312 can be 22 French.

In some examples, in the expanded configuration, the diameters of the inner layer 310 and outer layer 320 increase at the same ratio. For example, the inner layer 310 and outer layer 320 experience the same percentage of increase in diameter compared to their corresponding unexpanded diameters. In some examples, in the expanded configuration, the inner layer 310 and outer layer 320 increase at different ratios. For example, the inner layer 310 and outer layer 320 each experience a different percentage of increase in diameter compared to their corresponding unexpanded diameters. In some examples, in the expanded configuration, the diameter of the outer layer 320 increases by a greater ratio than the diameter of the inner layer 310, i.e., the diameter of the outer layer 320 increases by a greater percentage than the diameter of the inner layer 310. In some examples, in the expanded configuration, the diameter of the inner layer 310 increases at a greater ratio than the diameter of the outer layer 320, i.e., the diameter of the inner layer 310 increases by a greater percentage than the diameter of the outer layer 320.

In some examples, the inner layer 310 is composed of a different material than the outer layer 320, resulting in the inner layer 310 and outer layer 320 expanding at different ratios. In some examples, outer layer 320 is composed of a more elastic material than the inner layer 310 such that the diameter of the outer layer 320 increases at a greater ratio than the diameter of the inner layer 310. In some examples, the inner layer 310 is composed of a more clastic material than the outer layer 320 such that the diameter of the inner layer 310 increases at a greater ratio than the diameter of the outer layer 320.

As described herein, the fluid chamber 330 is inflated during expansion of the sheath 8 and deflated as the sheath 8 returns toward the unexpanded configuration. In the unexpanded configuration the fluid chamber 330 has a first volume and in the expanded configuration the fluid chamber 330 has a second, larger, volume. As illustrated in FIG. 29, an inflation fluid is provided to the fluid chamber 330 via the inflation port 340. The inflation port 340 can pass through the sheath hub 20 and/or be in fluid communication with a fluid port provided on the sheath hub 20. Example inflation fluids include a fluid, such as saline, or a gas.

The sheath 8 expands from the unexpanded configuration to the expanded configuration upon receipt of the inflation fluid in the fluid chamber 330 as shown, for example, by Ref. A in FIG. 29, identifying the direction of flow of the inflation fluid into the fluid chamber 330 during expansion of the sheath 8. The sheath 8 expands in response to the outwardly directed radial force exerted on the inner layer 310 and/or outer layer 320 from the increased volume of the inflation fluid received within the fluid chamber 330.

When the sheath 8 is deflated, the sheath 8 moves from the expanded configuration toward the unexpanded configuration upon withdrawal of the inflation fluid from the fluid chamber 330, i.e., the flow of inflation fluid toward and through the inflation port 340 in the direction opposite Ref. A in FIG. 29. The sheath 8 returns toward the unexpanded configuration in response a reduction in the outwardly directed radial force exerted on the inner layer 310 and/or outer layer 320 by the inflation fluid resulting from a decrease in the volume of the inflation fluid received within the fluid chamber 330.

In the unexpanded configuration, the fluid chamber 330 receives a first volume of the inflation fluid, and in the expanded configuration the fluid chamber receives a second, larger, volume of the inflation fluid. As a result, expansion of the fluid chamber 330/sheath 8 can be controlled by controlling the volume of inflation fluid received within the fluid chamber 330. For example, where a particular expanded diameter of the inner layer 310 and/or outer layer 320 is desired, a corresponding volume of inflation fluid is provided to the fluid chamber 330. Similarly, if a desired decrease in the diameter of the inner layer 310 and/or outer layer 320 is desired, a corresponding volume of inflation fluid is removed from the fluid chamber 330. The amount or volume of inflation fluid provided to or removed from the fluid chamber 330 can be pre-determined. In some examples, such pre-determined amount or volume can be changed by a user.

In addition to controlling the outer diameter of the sheath 8, the stiffness of the sheath 8 can be adjusted as desired based on patient anatomy and/or the size of the medical device being delivered. For example, the tight curvature of tortious vasculature structure may require the stiffness be adjusted (for example, increased) to partially straighten the curvature to allow passage of the medical device or (for example, decreased) to allow passage of the medical device through fragile tissue/vascular segment without additional expansion of the blood vessel caused by the combined diameter of an expanded sheath 8 and the medical device.

The stiffness of the sheath 8 can be controlled by adjusting the volume of inflation fluid provided to the fluid chamber 330. For example, an increase in the volume of the inflation fluid in the fluid chamber 330 results in a corresponding increase in the stiffness of the sheath 8.

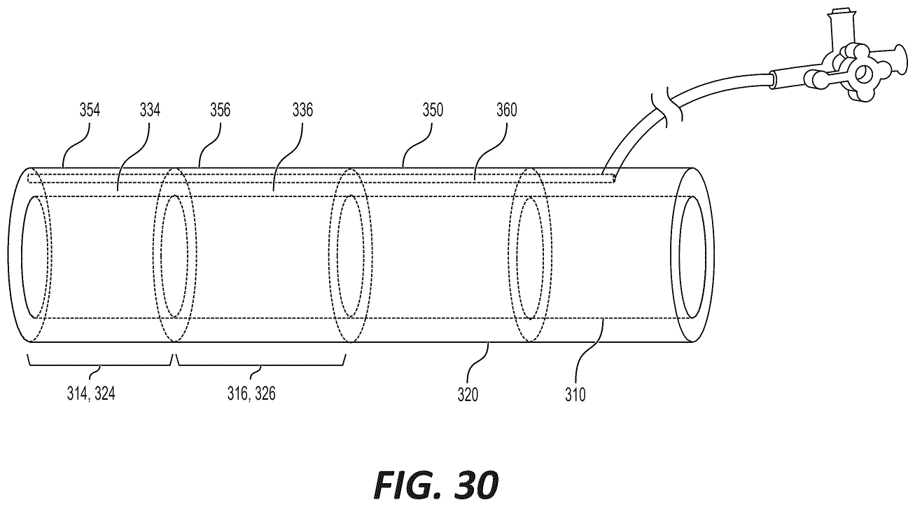

In some examples, the sheath 8 can include a plurality of tubular segments 350. FIG. 30 provides a partial cross-section view of the sheath 8 of FIG. 24 along section line C-C illustrating the plurality of tubular segments 350 provided along a length of the sheath 8. The number of tubular segments 350 can range from one to ten tubular segments 350.

The tubular segments 350 define a corresponding plurality of fluid chambers 330 between the inner layer 310 and the outer layer 320. The tubular segments 350 are coaxial along the length of the sheath 8. For example, FIG. 30 illustrates a first tubular segment 354 and second tubular segment 356 each defining a corresponding first fluid chamber 334 and a second fluid chamber 336. As provided in FIG. 30, the tubular segments 350 are arranged such that the distal end of the first tubular segment 354 is adjacent the proximal end of the second tubular segment 356. This arrangement is continued along the sheath 8.

The inflation port 340 is in fluid communication with the fluid chambers 330 of each of the plurality of tubular segments 350. In some examples, each of the plurality of tubular segments 350 are jointly connected to the inflation port 340. In some examples, the adjacent tubular segments 350 are not fluidly connected to each other. For example, each of the plurality of tubular segments 350 is separately connected to a corresponding one of a plurality of inflation ports 340 such that each of the tubular segments 350 are fluidly independent of each other. In some examples, each of the tubular segments 350 is separately connected to a corresponding one of a plurality of inflation ports 340.

As illustrated in FIG. 30, each of the tubular segments 350 is fluidly connected to the inflation port 340 by an inflation lumen 360. The inflation lumen 360 is provided between the outer surface of the outer layer 320 and the inner surface of the inner layers 310/central lumen 312. In some examples, the inflation lumen 360 is provided within the wall thickness of the inner layer 310 and/or outer layer 320 of the tubular segments 350. In some examples, the inflation lumen 360 passes though the fluid chambers 330 of each of the tubular segments 350. Where the tubular segments 350 are separately connected to a corresponding inflation port 340, the sheath 8 can include a separate inflation lumen 360 fluidly connecting the fluid chambers 330 with their corresponding inflation port 340. As a result, the sheath 8 can include a plurality of inflation lumens 360 passing between the inner layer 310 and outer layer 320.

Accordingly, the volume of inflation fluid received within each of the fluid chambers 330 can be varied along the length of the sheath 8. For example, in the unexpanded configuration, the fluid chamber 330 of each of the corresponding tubular segments 350 has a first volume of inflation fluid, in the expanded configuration the fluid chamber 330 of each of the corresponding tubular segments 350 has a second, larger, volume of inflation fluid.

The first (unexpanded) volume of each of the plurality of tubular segments 350 can vary from the first volume of at least one of another tubular segment 350 in the corresponding unexpanded configuration. Likewise, the second (expanded) volume of each of the plurality of tubular segments 350 can vary from the second volume of at least one of another tubular segment 350 in the corresponding expanded configuration. For example, in the expanded configuration, the first tubular segment 354 receives a volume of inflation fluid, and the second tubular segment 356 receives a second, different (for example, larger), volume of inflation fluid. Similarly, in the unexpanded configuration, the first tubular segment 354 contains a volume of inflation fluid, and the second tubular segment 356 receives a second, different, volume of inflation fluid.

The stiffness of each of the plurality of tubular segments 350 can vary in relation to the amount or volume of inflation fluid stored therein. For example, a tubular segment 350 having greater volume of inflation fluid is stiffer than a tubular segment 350 having a smaller volume of inflation fluid. By varying the amount of inflation fluid within each of the tubular segments 350 the stiffness of the sheath 8 can be varied along its length.

In some examples, the volume of inflation fluid introduced into and/or withdrawn from each of the plurality of tubular segments 350 is separately controlled, for example, by a mechanically or electrically controlled pump, including a computer controlled electrical pump. For example, the volume of inflation fluid introduced into each of the plurality of tubular segments 350 is separately controlled to vary the expansion and/or stiffness of the tubular segments 350. In some examples, the volume of inflation fluid introduced into each of the plurality of tubular segments 350 is controlled to incrementally expand the sheath 8, i.e., incrementally expand the inner layer 310 and outer layer 320 along a length of the sheath 8. In some examples, the volume of inflation fluid introduced into each into each of the plurality of tubular segments 350 is controlled to incrementally expand the strain relief layer 26 of the sheath 8.

In some examples, the inner layer 310 and the outer layer 320 of the sheath 8 are composed of the same material. In some examples, the expansion and stiffness of the sheath 8 can be controlled by varying the material of the inner layer 310 and/or outer layer 320 along a length of the sheath 8. The inner layer 310 and the outer layer 320 are composed of an elastomeric material. Example materials include silicon, polyimide, polytetrafluoroethylene (PTFE), expanded polytetrafluoroethylene (ePTFE), ethylene tetrafluoroethylene (ETFE), nylon, polyethylene, polyamide, polyether block amide (for example, Pebax), and/or combinations thereof.

In an example sheath 8, the expansion ratios between the inner layer 310 and the outer layer 320 of the sheath 8 can be varied by varying the materials used for the inner and outer layers 310, 320 along a corresponding tubular segment 350/length of the sheath 8. Similarly, the stiffness or elasticity of the sheath 8 can be varied along the sheath 8 by varying the materials used for the inner layer 310 and outer layer 320 along a corresponding tubular segment 350/length of the sheath 8.

In some examples, the inner layer 310 includes a plurality of segments of varying stiffness or elasticity. The plurality of segments can include a first segment 314 having a first stiffness (or a first elasticity) and a second segment 316 having a second, different, stiffness (for example, a second different elasticity). For example, at least one of the plurality of segments of the inner layer 310 is composed of a different material than another one of the plurality of segments of the inner layer 310 having a different stiffness or elasticity, i.e., the first segment 314 and second segment 316 of the inner layer 310 are composed of different materials of varying stiffness or elasticity.

In another example, the outer layer 320 includes a plurality of segments of varying stiffness or elasticity, the plurality of segments including a first segment 324 having a first stiffness (or a first elasticity) and a second segment 326 having a second, different, stiffness (for example, second different elasticity). For example, at least one of the plurality of segments of the outer layer 320 is composed of a different material than another one of the plurality of segments of the outer layer 320 having a different stiffness or elasticity, i.e., the first segment 314 and second segment 316 of the outer layer 320 are composed of different materials of varying stiffness or elasticity.

It is contemplated that the materials of the inner and outer layers 310, 320 can vary between each other in a given segment along the sheath 8. In some examples, the materials of the inner layer 310 and outer layer 320 can vary between adjacent segments along the sheath 8.

A method of expanding an inflatable introducer sheath 8 for delivering a medical device (for example, to a procedure site) is described herein. A sheath 8 is provided including an inner layer 310 defining a central lumen 312 extending therethrough and an outer layer 320 provided over the inner layer 310 such that a fluid chamber 330 is defined therebetween.

The sheath 8 is inserted at least partially into the blood vessel of the patient and the distal end of the sheath 8 is positioned at a location proximate the treatment site. In some examples, the sheath 8 is partially expanded before being inserted into the patient. For example, a first volume of inflation fluid is introduced into the fluid chamber 330 before inserting the sheath 8 at least partially into the blood vessel of the patient. The introduction of the first volume of the inflation fluid increases a volume of the fluid chamber 330 and partially expands at least a portion of the sheath 8 from the unexpanded configuration to the expanded configuration, thereby increasing the stiffness of the sheath 8 to aid in insertion. With the stiffness of the sheath 8 increased, the partially expanded sheath 8 is then inserted into the patient's blood vessel.

Once in position at the treatment site, the inflation fluid is introduced into the fluid chamber 330 by an inflation port 340 in fluid communication with the fluid chamber 330. At least a portion of the sheath 8 (for example, inner layer 310 and/or outer layer 320) is thereby expanded from an initial (for example, unexpanded or partially expanded) configuration in which the central lumen 312 has a first diameter, to an expanded configuration in which the central lumen 312 has a second, larger, diameter.

The sheath 8 expands from the unexpanded configuration to the expanded configuration upon receipt of the inflation fluid in the fluid chamber 330. For example, the sheath 8 expands in response to the outwardly directed radial force exerted on the inner layer 310 and/or outer layer 320 in response to the increased volume of the inflation fluid received within the fluid chamber 330. Similarly, the sheath 8 moves from the expanded configuration toward the unexpanded configuration upon withdrawal of the inflation fluid from the fluid chamber 330. For example, the sheath 8 returns toward the unexpanded configuration in response a reduction in the outwardly directed radial force exerted on the inner layer 310 and/or outer layer 320 by the inflation fluid resulting from a decrease in the volume of the inflation fluid received within the fluid chamber 330.

As described herein, the sheath 8 may include a plurality of tubular segments 350 defining corresponding plurality of fluid chambers 330 (for example, first fluid chamber 334, second fluid chamber 336) between the inner layer 310 and outer layer 320. The stiffness and/or inflation ratios of inner and/or outer layers 310, 320 of various segments of the sheath 8 can be varied by varying the volume of the inflation fluid introduced into of the plurality of tubular segments 350, such that a volume of the inflation fluid introduced into one of the plurality of tubular segments 350 varies from at least one of another tubular segment 350. As a result, in the expanded configuration, the stiffness of each of the plurality of tubular segments 350 varies in relation to their corresponding volume of inflation fluid.

In some examples, the volume of inflation fluid introduced into and/or withdrawn from each of the plurality of tubular segments 350 is separately controlled. For example, the volume of inflation fluid introduced into each of the plurality of tubular segments 350 is controlled to vary the expansion of the tubular segments 350 (for example, the outer layer 320 and/or inner layer 310/central lumen 312). In some examples, the volume of inflation fluid introduced into each of the plurality of tubular segments 350 is controlled to incrementally expand the sheath 8 (for example, incrementally expand the inner layer 310 and outer layer 320 along a length of the sheath 8).

As the sheath 8 is expanded, the central lumen 312 of the sheath expands allowing a medical device can be inserted at lower push force than required for a non-expanded sheath. Expansion of the sheath 8 also results in a corresponding expansion of the strain relief layer 26.

The medical device is advanced through the central lumen 312 and advanced beyond the distal opening in the sheath 8 to a treatment site within the blood vessel. In some examples, the medical device is contracted or compressed radially as it passes through the strain relief layer 26, from the proximal portion 242, through the tapered portion 248 and into the smaller diameter distal portion 246. In some examples, advancing the medical device through the central lumen 312 of the sheath 8 includes causing the sheath 8 to locally expand from the unexpanded configuration to the expanded configuration at a location proximate the medical device in response to the outwardly directed radially force of the medical device exerted against the sheath 8 (for example, inner layer 310). In some examples, as the medical device passes through the corresponding portion of the lumen of sheath 8, the sheath 8 and strain relief layer 26 locally contracts towards the unexpanded configuration.

The medical device is then passed through the distal opening of the sheath 8 and delivered to the treatment site. The position of the medical device can be moved or adjusted until the medical device is adequately positioned within the patient.

With the medical device delivered to the treatment site, any delivery system/components coupled to the medical device are then removed from the medical device and withdrawn from/through the central lumen 312 of the sheath 8. In some examples, withdrawing the medical device from the central lumen 312 of the sheath 8 further includes locally contracting the sheath 8 at least partially back toward the unexpanded configuration as the medical device passes through the central lumen 312, for example, by the radially inward force of an elastic outer layer 250/outer jacket provided over the sheath 8.

The sheath 8 can then be deflated by withdrawing the inflation fluid from the fluid chamber 330 by the inflation port 340. At least a portion the sheath 8 (for example, the inner layer 310 and/or outer layer 320) is thereby moved toward the unexpanded configuration. In some examples, the sheath 8 includes an elastic outer layer 250 providing a radially inward force that assists in directing the sheath 8 (and strain relief layer 26) to/towards the unexpanded configuration.

The sheath 8 is then withdrawn from the patient's blood vessel and the opening in the blood vessel and skin closed.

In some examples, the inflatable sheath 8 of FIGS. 24-30, can used to expand the layered sheath as described in reference to FIGS. 11-23 including at least one of the inner layer and/or outer layer includes at least one folded portion, for example, ridges 126 and valleys 128 of the fourth (for example, outer) layer 108 of the sheath 8 illustrated in FIGS. 11-14, and folded portion 218 of the inner layer 202 of the sheath 8 illustrated in FIGS. 15-23. Locally expanding the lumen of the layered sheath causes a length of the folded portion to at least partially unfold. Similarly, locally contracting the sheath 8 at least partially back to the unexpanded configuration causes a length of the folded portion to urge back towards a folded configuration.

In some examples as described herein, the outer layer is a discontinuous outer layer and includes an overlapping portion (for example, overlapping portion 220) and an underlying portion (for example, underlying portion 222). When the layered sheath is in the unexpanded configuration, the overlapping portion overlaps the underlying portion with the folded portion of the inner layer disposed between the overlapping portion and the underlying portion (FIGS. 17, 19, 22, 23). As the layered sheath locally expands to/toward the expanded configuration, a length of the overlapping portion moves circumferentially with respect to the underlying portion unfolding. As illustrated in FIG. 20, when the layered sheath is fully expanded, the inner layer extends into the annular gap 232 formed between the longitudinal edges of the overlapping portion 220 and the underlying portion 222 of the outer layer 204.

In some examples, the sheath 8 includes an elastic outer layer 250 that extends at least partially over the outer layer and/or the strain relief layer 26. The elastic outer layer 250 locally expands and contracts as the medical device is advanced through the lumen of the sheath 8. In some examples, the clastic outer layer 250 urges the various layers of the sheath 8 toward an unexpanded configuration.

The medical device described herein can include a prosthetic device mounted in a radially crimped state on a delivery apparatus, and the act of advancing the prosthetic device through the lumen of the sheath 8 comprises advancing the delivery apparatus and the prosthetic device through lumen of the sheath 8 and into the vasculature of the patient. In some examples, the prosthetic device comprises a prosthetic heart valve and the method further comprises implanting the prosthetic heart valve at a treatment site within the patient. As described herein, the prosthetic heart valve is mounted on a balloon catheter of the delivery apparatus as the prosthetic heart valve is advanced through the sheath 8.

### Exemplary Aspects

In view of the described processes and compositions, herein are described certain more particularly described aspects of the disclosures. These particularly recited aspects should not, however, be interpreted to have any limiting effect on any different claims containing different or more general teachings described herein, or that the “particular” aspects are somehow limited in some way other than the inherent meanings of the language and formulas literally used therein.

EXAMPLE 1. An inflatable introducer sheath for delivering a medical device, the sheath including: an inner layer defining a central lumen extending therethrough; an outer layer provided over the inner layer such that a fluid chamber is defined therebetween; and an inflation port in fluid communication with the fluid chamber, wherein, upon inflation of the fluid chamber, at least a portion of the sheath expands from an unexpanded configuration in which the central lumen has a first diameter to an expanded configuration in which the central lumen has a second, larger, diameter, wherein, upon deflation of the fluid chamber, at least a portion of the sheath returns toward the unexpanded configuration.

EXAMPLE 2. The sheath according to any example herein, particularly example 1, wherein, in the expanded configuration, the inner and outer layers each expand from a first diameter to a second, larger, diameter.

EXAMPLE 3. The sheath according to any example herein, particularly examples 1-2, wherein, in the expanded configuration, diameters of the inner and outer layers increase at the same ratio.

EXAMPLE 4. The sheath according to any example herein, particularly examples 1-2, wherein, in the expanded configuration, diameters of the inner and outer layers increase at different ratios.

EXAMPLE 5. The sheath according to any example herein, particularly example 4, wherein, in the expanded configuration, the diameter of the outer layer increases at a greater ratio than the diameter of the inner layer.

EXAMPLE 6. The sheath according to any example herein, particularly example 4, wherein, in the expanded configuration, the diameter of the inner layer increases at a greater ratio than the diameter of the outer layer.

EXAMPLE 7. The sheath according to any example herein, particularly examples 1-6, wherein the inner layer is composed of a different material than the outer layer such that in the expanded configuration, the diameters of the inner and outer layers increase at different ratios.

EXAMPLE 8. The sheath according to any example herein, particularly example 7, wherein the outer layer is composed of a more elastic material than the inner layer such that the diameter outer layer increases at a greater ratio than the diameter of the inner layer.

EXAMPLE 9. The sheath according to any example herein, particularly example 7, wherein the inner layer is composed of a more elastic material than the outer layer such that the diameter of the inner layer increases at a greater ratio than the diameter of the outer layer.

EXAMPLE 10. The sheath according to any example herein, particularly examples 1-9, wherein, in the unexpanded configuration, the fluid chamber has a first volume and in the expanded configuration the fluid chamber has a second, larger, volume.

EXAMPLE 11. The sheath according to any example herein, particularly examples 1-10, further comprising an inflation fluid provided to the fluid chamber via the inflation port, wherein the sheath expands from the unexpanded configuration to the expanded configuration upon receipt of the inflation fluid in the fluid chamber, wherein the sheath moves from the expanded configuration toward the unexpanded configuration upon withdrawal of the inflation fluid from the fluid chamber.

EXAMPLE 12. The sheath according to any example herein, particularly examples 1-11, further comprising an inflation fluid provided to the fluid chamber via the inflation port, wherein, in the unexpanded configuration, the fluid chamber receives a first volume of the inflation fluid, and in the expanded configuration the fluid chamber receives a second, larger, volume of the inflation fluid.

EXAMPLE 13. The sheath according to any example herein, particularly examples 1-12, wherein the inner layer and the outer layer are composed of an elastomeric material, wherein a stiffness or elasticity of the sheath in the expanded and/or unexpanded configuration is constant along a length of the sheath.

EXAMPLE 14. The sheath according to any example herein, particularly examples 1-13, wherein the inner layer and the outer layer are composed of an elastomeric material, wherein a stiffness or elasticity of the sheath in the expanded and/or unexpanded configuration varies along a length of the sheath.

EXAMPLE 15. The sheath according to any example herein, particularly example 14, wherein the inner layer includes a plurality of segments of varying stiffness or elasticity, the plurality of segments including a first segment having a first stiffness and a second segment having a second, different, stiffness.

EXAMPLE 16. The sheath according to any example herein, particularly example 15, wherein at least one of the plurality of segments of the inner layer is composed of a different material than an other one of the plurality of segments of the inner layer having a different stiffness or elasticity.

EXAMPLE 17. The sheath according to any example herein, particularly examples 14-16, wherein the outer layer includes a plurality of segments of varying stiffness or elasticity, the plurality of segments including a first segment having a first stiffness and a second segment having a second, different, stiffness.

EXAMPLE 18. The sheath according to any example herein, particularly example 17, wherein at least one of the plurality of segments of the outer layer is composed of a different material than an other one of the plurality of segments of the outer layer having a different stiffness or elasticity.

EXAMPLE 19. The sheath according to any example herein, particularly examples 1-18, wherein the sheath includes a plurality tubular segments defining corresponding plurality of fluid chambers between the inner and outer layers.

EXAMPLE 20. The sheath according to any example herein, particularly example 19,

wherein the corresponding inner and outer layers of each of the plurality of tubular segments is composed of materials having different stiffness or elasticity.

EXAMPLE 21. The sheath according to any example herein, particularly examples 19-20, wherein the inflation port is in fluid communication with the fluid chambers of each of the plurality of tubular segments.

EXAMPLE 22. The sheath according to any example herein, particularly examples 19-21, wherein each of the plurality of tubular segments is separately connected to the inflation port.

EXAMPLE 23. The sheath according to any example herein, particularly examples 19-21, wherein each of the plurality of tubular segments is separately connected to a corresponding one of a plurality of inflation ports.

EXAMPLE 24. The sheath according to any example herein, particularly examples 19-23, wherein each of the tubular segments is fluidly connected to the inflation port by an inflation lumen.

EXAMPLE 25. The sheath according to any example herein, particularly examples 19-23, wherein each of the tubular segments is fluidly connected to a respective inflation port by a corresponding inflation lumen.

EXAMPLE 26. The sheath according to any example herein, particularly examples 19-25, wherein, in the unexpanded configuration, the fluid chamber of each of the corresponding tubular segments has a first volume, wherein, in the expanded configuration, the fluid chamber of each of the corresponding tubular segments has a second, larger, volume.

EXAMPLE 27. The sheath according to any example herein, particularly example 26, wherein the volume of each of the plurality of tubular segments varies from at least one of an other tubular segment.

EXAMPLE 28. The sheath according to any example herein, particularly example 26, wherein, in the expanded configuration, the stiffness of each of the plurality of tubular segments varies in relation to their corresponding volume.

EXAMPLE 29. The sheath according to any example herein, particularly examples 26-28, further comprising an inflation fluid provided to the fluid chamber via the inflation port, wherein the volume of inflation fluid introduced into and/or withdrawn from each of the plurality of tubular segments is separately controlled.

EXAMPLE 30. The sheath according to any example herein, particularly example 29, wherein the volume of inflation fluid introduced into each of the plurality of tubular segments is separately controlled to vary the expansion of the tubular segments.

EXAMPLE 31. The sheath according to any example herein, particularly examples 29-30, wherein the volume of inflation fluid introduced into each of the plurality of tubular segments is controlled to incrementally expand the sheath.

EXAMPLE 32. The sheath according to any example herein, particularly examples 29-31, wherein the volume of inflation fluid introduced into and/or withdrawn from each of the plurality of tubular segments is separately controlled by a processor.

EXAMPLE 33. The sheath according to any example herein, particularly examples 1-32, wherein, in the expanded configuration, at least a portion of the sheath is configured to locally expand from second diameter to a third, larger, diameter, and then locally contract at least partially back to the second diameter.

EXAMPLE 34. The sheath according to any example herein, particularly examples 1-33, further including a tubular strain relief layer provided over the inner layer positioned at a proximal end of the sheath and extending along at least a portion of a length of the sheath, wherein the strain relief layer comprises a stiffer and/or less elastomeric material than the inner layer and/or outer layers that restricts expansion of the inner and outer layers.

EXAMPLE 35. The sheath according to any example herein, particularly example 34, wherein the strain relief layer 26 includes: a proximal portion adjacent a proximal end of the strain relief layer; a distal portion adjacent a distal end of the strain relief layer; and a tapered portion extending between the distal portion and the proximal portion, wherein a diameter of the proximal portion is greater than a diameter of the distal portion.

EXAMPLE 36. The sheath according to any example herein, particularly examples 34-35, wherein at least a portion of the strain relief layer is configured to locally expand from an unexpanded configuration at a first diameter to an expanded configuration at a second diameter, and then locally contract at least partially back to the unexpanded configuration.

EXAMPLE 37. The sheath according to any example herein, particularly examples 34-36, wherein the strain relief layer comprises a material having a higher durometer than at least one of the inner layer and/or the outer layer.

EXAMPLE 38. The sheath according to any example herein, particularly examples 34-37, wherein the strain relief layer comprises polyurethane.

EXAMPLE 39. The sheath according to any example herein, particularly examples 1-38, further including: an outer introducer sheath provided over the outer layer, the outer introducer sheath movable along the outer layer, outer introducer sheath comprising: a continuous first layer defining a lumen extending therethrough, the inner layer having at least one folded portion; and a second layer provided over the first layer, where the second layer is discontinuous and includes an overlapping portion and an underlying portion, and the overlapping portion overlaps the underlying portion, wherein the outer introducer sheath is configured to locally expand from an unexpanded configuration in the which the lumen has a first diameter to an expanded configuration in which the lumen has a second diameter that is larger than the first diameter, and then locally contract at least partially back to the unexpanded configuration.

EXAMPLE 40. The sheath according to any example herein, particularly example 39, wherein, when in the unexpanded configuration, the folded portion extends circumferentially over an outer surface of the inner layer and/or outer layer, wherein at least a portion of the folded portion of the inner layer is positioned between the overlapping an underlying portions.

EXAMPLE 41. The sheath according to any example herein, particularly examples 39-40, wherein in the expanded configuration local expansion causes a length of the folded portion to at least partially unfold, wherein in the expanded configuration local expansion of the sheath causes a length of the overlapping portion to move circumferentially with respect to the underlying portion, wherein in the expanded configuration, local expansion of the sheath forms a gap between longitudinally extending edges of the outer layer, wherein at least a portion of the unfolded portion extends into the gap.

EXAMPLE 42. The sheath according to any example herein, particularly examples 1-41, further including an elastic outer jacket extending at least partially over the sheath where the outer cover locally expands and contracts as the sheath moves between the expanded and unexpanded configurations, wherein the elastic outer cover exerts a radially inward force on the sheath.

EXAMPLE 43. A method of expanding an inflatable introducer sheath, the method including: providing the sheath including an inner layer defining a central lumen extending therethrough and an outer layer provided over the inner layer such that a fluid chamber is defined therebetween; introducing an inflation fluid into the fluid chamber by an inflation port in fluid communication with the fluid chamber thereby expanding at least a portion of the sheath from an unexpanded configuration in which the central lumen has a first diameter to an expanded configuration in which the central lumen has a second, larger, diameter; and withdrawing the inflation fluid from the fluid chamber by the inflation port thereby moving at least a portion of the sheath toward the unexpanded configuration.

EXAMPLE 44. The method according to any example herein, particularly example 43, wherein the sheath expands from the unexpanded configuration to the expanded configuration upon receipt of the inflation fluid in the fluid chamber, wherein the sheath moves from the expanded configuration toward the unexpanded configuration upon withdrawal of the inflation fluid from the fluid chamber.

EXAMPLE 45. The method according to any example herein, particularly examples 43-45, wherein the sheath includes a plurality of tubular segments defining corresponding plurality of fluid chambers the inner and outer layers, wherein introducing an inflation fluid into the fluid chamber further includes: varying the volume of the inflation fluid introduced into of the plurality of tubular segments, such that a volume of the inflation fluid introduced into one of the plurality of tubular segments varies from at least one of an other tubular segment, wherein, in the expanded configuration, the stiffness of each of the plurality of tubular segments varies in relation to their corresponding volume of inflation fluid.

EXAMPLE 46. The method according to any example herein, particularly example 45, wherein the volume of inflation fluid introduced into and/or withdrawn from each of the plurality of tubular segments is separately controlled.

EXAMPLE 47. The method according to any example herein, particularly examples 45-46, The sheath of claims 26-29, wherein the volume of inflation fluid introduced into each of the plurality of tubular segments is controlled to vary the expansion of the tubular segments.

EXAMPLE 48. The method according to any example herein, particularly examples 45-47, wherein the volume of inflation fluid introduced into each of the plurality of tubular segments is controlled to incrementally expand the sheath.

EXAMPLE 49. A method of delivering a medical device through a sheath including: providing the sheath including an inner layer defining a central lumen extending therethrough and an outer layer provided over the inner layer such that a fluid chamber is defined therebetween; introducing an inflation fluid into the fluid chamber by an inflation port in fluid communication with the fluid chamber thereby expanding at least a portion of the sheath from an unexpanded configuration in which the central lumen has a first diameter to an expanded configuration in which the central lumen has a second, larger, diameter; introducing a medical device into the central lumen of the sheath; advancing the medical device through the central lumen of the sheath; withdrawing the medical device from the central lumen of the sheath; and withdrawing the inflation fluid from the fluid chamber by the inflation port thereby moving at least a portion the sheath toward the unexpanded configuration.

EXAMPLE 50. The method according to any example herein, particularly example 49, wherein the sheath includes a plurality of tubular segments defining corresponding plurality of fluid chambers between the inner and outer layers, wherein introducing an inflation fluid into the fluid chamber further includes: varying the volume of the inflation fluid introduced into of the plurality of tubular segments, such that a volume of the inflation fluid introduced into one of the plurality of tubular segments varies from at least one of an other tubular segment, wherein, in the expanded configuration, the stiffness of each of the plurality of tubular segments varies in relation to their corresponding volume of inflation fluid.

EXAMPLE 51. A method of inserting a medical device into a blood vessel of a patient, the method including: providing the sheath including an inner layer defining a central lumen extending therethrough and an outer layer provided over the inner layer such that a fluid chamber is defined therebetween; inserting the sheath at least partially into the blood vessel of the patient; introducing an inflation fluid into the fluid chamber by an inflation port in fluid communication with the fluid chamber thereby expanding at least a portion of the sheath from an unexpanded configuration in which the central lumen has a first diameter to an expanded configuration in which the central lumen has a second, larger, diameter; introducing the medical device into the central lumen of the sheath; advancing the medical device through the central lumen of the sheath; advancing the medical device beyond a distal opening in the sheath to a treatment site within the blood vessel; withdrawing the medical device from the central lumen of the sheath; withdrawing the inflation fluid from the fluid chamber by the inflation port thereby moving at least a portion the sheath 8 toward the unexpanded configuration; and removing the sheath from the blood vessel of the patient.

EXAMPLE 52. The method according to any example herein, particularly example 51, wherein the sheath expands from the unexpanded configuration to the expanded configuration upon receipt of the inflation fluid in the fluid chamber, wherein the sheath moves from the expanded configuration toward the unexpanded configuration upon withdrawal of the inflation fluid from the fluid chamber.

EXAMPLE 53. The method according to any example herein, particularly examples 51-52, wherein the sheath includes a plurality of tubular segments defining corresponding plurality of fluid chambers between the inner and outer layers, wherein introducing an inflation fluid into the fluid chamber further includes: varying the volume of the inflation fluid introduced into of the plurality of tubular segments, such that a volume of the inflation fluid introduced into one of the plurality of tubular segments varies from at least one of an other tubular segment, wherein, in the expanded configuration, the stiffness of each of the plurality of tubular segments varies in relation to their corresponding volume of inflation fluid.

EXAMPLE 54. The method according to any example herein, particularly examples 51-53, wherein a first volume of inflation fluid is introduced into the fluid chamber before inserting the sheath at least partially into the blood vessel of the patient, wherein the first volume of the inflation fluid increases a volume of the fluid chamber, wherein at least a portion of the sheath is partially expanded the unexpanded configuration toward the expanded configuration increasing the stiffness of the sheath.

EXAMPLE 55. The method according to any example herein, particularly examples 51-54, wherein advancing the medical device through the central lumen of the sheath further includes causing the sheath to locally expand from the unexpanded configuration to the expanded configuration at a location proximate the medical device in response to the outwardly directed radially force of the medical device exerted against the sheath, wherein withdrawing the medical device from the central lumen of the sheath further includes locally contracting the sheath at least partially back to the unexpanded configuration as the medical device passes through the central lumen.

EXAMPLE 56. The method according to any example herein, particularly examples 51-55 further including: withdrawing the medical device from the central lumen of the sheath; and withdrawing the inflation fluid from the fluid chamber by the inflation port thereby moving at least a portion of the sheath toward the unexpanded configuration.

EXAMPLE 57. The method according to any example herein, particularly examples 51-56, wherein the medical device is a prosthetic device mounted in a radially crimped state on a delivery apparatus, and the act of advancing the prosthetic device through the lumen of the sheath comprises advancing the delivery apparatus and the prosthetic device through lumen of the sheath and into the vasculature of the patient.

EXAMPLE 58. The method according to any example herein, particularly example 57, wherein the prosthetic device comprises a prosthetic heart valve and the method further comprises implanting the prosthetic heart valve at a treatment site within the patient.

EXAMPLE 59. The method according to any example herein, particularly example 58, wherein the prosthetic heart valve is mounted on a balloon catheter of the delivery apparatus as the prosthetic heart valve is advanced through the sheath.

EXAMPLE 60. The method according to any example herein, particularly examples 51-59, wherein the sheath is inserted into a femoral artery of a patient.

In view of the many possible aspects to which the principles of the disclosed disclosure can be applied, it should be recognized that the illustrated examples are only preferred examples of the disclosure and should not be taken as limiting the scope of the disclosure. Rather, the scope of the disclosure is defined by the following claims. We, therefore, claim as our disclosure all that comes within the scope and spirit of these claims.

1. An inflatable introducer sheath for delivering a medical device, the sheath including:
an inner layer defining a central lumen extending therethrough;
an outer layer provided over the inner layer such that a fluid chamber is defined therebetween; and
an inflation port in fluid communication with the fluid chamber,
wherein, upon inflation of the fluid chamber, at least a portion of the sheath expands from an unexpanded configuration in which the central lumen has a first diameter to an expanded configuration in which the central lumen has a second, larger, diameter,
wherein, upon deflation of the fluid chamber, at least a portion of the sheath returns toward the unexpanded configuration.

2. The sheath of claim 1, wherein, in the expanded configuration, the inner and outer layers each expand from a first diameter to a second, larger, diameter,
wherein, in the unexpanded configuration, the fluid chamber has a first volume and in the expanded configuration the fluid chamber has a second, larger, volume.

3. The sheath of claim 1, further comprising an inflation fluid provided to the fluid chamber via the inflation port,
wherein the sheath expands from the unexpanded configuration to the expanded configuration upon receipt of the inflation fluid in the fluid chamber,
wherein the sheath moves from the expanded configuration toward the unexpanded configuration upon withdrawal of the inflation fluid from the fluid chamber.

4. The sheath of claim 1, further comprising an inflation fluid provided to the fluid chamber via the inflation port,
wherein, in the unexpanded configuration, the fluid chamber receives a first volume of the inflation fluid, and in the expanded configuration the fluid chamber receives a second, larger, volume of the inflation fluid.

5. The sheath of claim 1, wherein the inner layer and the outer layer are composed of an elastomeric material,
wherein a stiffness or elasticity of the sheath in the expanded and/or unexpanded configuration varies along a length of the sheath.

6. The sheath of claim 5, wherein the inner layer includes a plurality of segments of varying stiffness or elasticity, the plurality of segments including a first segment having a first stiffness and a second segment having a second, different, stiffness,
wherein the outer layer includes a plurality of segments of varying stiffness or elasticity, the plurality of segments including a first segment having a first stiffness and a second segment having a second, different, stiffness.

7. The sheath of claim 1, wherein the sheath includes a plurality of tubular segments defining corresponding plurality of fluid chambers between the inner and outer layers.

8. The sheath of claim 7, wherein the corresponding inner and outer layers of each of the plurality of tubular segments is composed of materials having different stiffness or elasticity.

9. The sheath of claim 7, wherein, in the unexpanded configuration, the fluid chamber of each of the corresponding tubular segments has a first volume,
wherein, in the expanded configuration, the fluid chamber of each of the corresponding tubular segments has a second, larger, volume.

10. The sheath of claim 9, wherein the volume of each of the plurality of tubular segments varies from at least one of an other tubular segment,
wherein, in the expanded configuration, a stiffness of each of the plurality of tubular segments varies in relation to their corresponding volume.

11. The sheath of claim 9, further comprising an inflation fluid provided to the fluid chamber via the inflation port, wherein the volume of inflation fluid introduced into and/or withdrawn from each of the plurality of tubular segments is separately controlled such that the volume of inflation fluid introduced into each of the plurality of tubular segments is separately controlled to vary the expansion of each of the plurality of tubular segments.

12. The sheath of claim 1, wherein, in the expanded configuration, the inner and outer layers each expand from a first diameter to a second, larger, diameter,
wherein, in the expanded configuration, at least a portion of the sheath is configured to locally expand from the second diameter to a third, larger, diameter, and then locally contract at least partially back to the second diameter.

13. The sheath of claim 1, further including a tubular strain relief layer provided over the inner layer, the tubular strain relief layer positioned at a proximal end of the sheath and extending along at least a portion of a length of the sheath,
wherein the strain relief layer comprises a stiffer and/or less elastomeric material than the inner layer and/or outer layers that restricts expansion of the inner and outer layers.

14. The sheath of claim 1, further including:
an outer introducer sheath provided over the outer layer, the outer introducer sheath movable along the outer layer, outer introducer sheath comprising:
a continuous first layer defining a lumen extending therethrough, the inner layer having at least one folded portion; and
a second layer provided over the first layer, where the second layer is discontinuous and includes an overlapping portion and an underlying portion, and the overlapping portion overlaps the underlying portion,
wherein the outer introducer sheath is configured to locally expand from an unexpanded configuration in the which the lumen has a first diameter to an expanded configuration in which the lumen has a second diameter that is larger than the first diameter, and then locally contract at least partially back to the unexpanded configuration.

15. A method of expanding an inflatable introducer sheath, the method including:
providing the sheath including an inner layer defining a central lumen extending therethrough and an outer layer provided over the inner layer such that a fluid chamber is defined therebetween;
introducing an inflation fluid into the fluid chamber by an inflation port in fluid communication with the fluid chamber thereby expanding at least a portion of the sheath from an unexpanded configuration in which the central lumen has a first diameter to an expanded configuration in which the central lumen has a second, larger, diameter; and
withdrawing the inflation fluid from the fluid chamber by the inflation port thereby moving at least a portion of the sheath toward the unexpanded configuration.

16. The method of claim 15, wherein the sheath expands from the unexpanded configuration to the expanded configuration upon receipt of the inflation fluid in the fluid chamber,
wherein the sheath moves from the expanded configuration toward the unexpanded configuration upon withdrawal of the inflation fluid from the fluid chamber.

17. The method of claim 15, wherein the sheath includes a plurality of tubular segments defining corresponding plurality of fluid chambers the inner and outer layers,
wherein introducing an inflation fluid into the fluid chamber further includes varying the volume of the inflation fluid introduced into of the plurality of tubular segments, such that a volume of the inflation fluid introduced into one of the plurality of tubular segments varies from at least one of an other tubular segment,
wherein, in the expanded configuration, a stiffness of each of the plurality of tubular segments varies in relation to their corresponding volume of inflation fluid.

18. The method of claim 17, wherein the volume of inflation fluid introduced into each of the plurality of tubular segments is controlled to vary the expansion of the tubular segments.

19. A method of delivering a medical device through a sheath including:
providing the sheath including an inner layer defining a central lumen extending therethrough and an outer layer provided over the inner layer such that a fluid chamber is defined therebetween;
introducing an inflation fluid into the fluid chamber by an inflation port in fluid communication with the fluid chamber thereby expanding at least a portion of the sheath from an unexpanded configuration in which the central lumen has a first diameter to an expanded configuration in which the central lumen has a second, larger, diameter;
introducing a medical device into the central lumen of the sheath;
advancing the medical device through the central lumen of the sheath;
withdrawing the medical device from the central lumen of the sheath; and
withdrawing the inflation fluid from the fluid chamber by the inflation port thereby moving at least a portion the sheath toward the unexpanded configuration.

20. The method of claim 19, wherein the sheath includes a plurality of tubular segments defining corresponding plurality of fluid chambers between the inner and outer layers,
wherein introducing an inflation fluid into the fluid chamber further includes:
varying the volume of the inflation fluid introduced into of the plurality of tubular segments, such that a volume of the inflation fluid introduced into one of the plurality of tubular segments varies from at least one of an other tubular segment,
wherein, in the expanded configuration, a stiffness of each of the plurality of tubular segments varies in relation to their corresponding volume of inflation fluid.
